SEQUENCE LISTING (22 sequences)

SEQ ID NO: 1 - protein, 661 aa, Mus musculus
MELEVPDEAESAEAGAVTAEAAWSAESGAAAGMTQKKAGLAEAPLVTGQPGPGHGKKLGHRGVDASGETTYKKTTSSTLKGAIQLGIGYTVGNLSSKPERDVLMQDFYVVESIFFPSEGSNLTPAHHFQDFRFKTYAPVAFRYFRELFGIRPDDYLYSLCNEPLIELSNPGASGSVFYVTSDDEFIIKTVMHKEAEFLQKLLPGYYMNLNQNPRTLLPKFYGLYCVQSGGKNIRVVVMNNVLPRVVKMHLKFDLKGSTYKRRASKKEKEKSLPTYKDLDFMQDMPEGLLLDSDTFGALVKTLQRDCLVLESFKIMDYSLLLGVHNIDQQERERQAEGAQSKADEKRPVAQKALYSTAMESIQGGAARGEAIETDDTMGGIPAVNGRGERLLLHIGIIDILQSYRFIKKLEHTWKALVHDGDTVSVHRPSFYAERFFKFMSSTVFRKSSSLKSSPSKKGRGALLAVKPLGPTAAFSASQIPSEREDVQYDLRGARSYPTLEDEGRPDLLPCTPPSFEEATTASIATTLSSTSLSIPERSPSDTSEQPRYRRRTQSSGQDGRPQEEPHAEDLQKITVQVEPVCGVGVVPKEEGAGVEVPPCGASAAASVEIDAASQASEPASQASDEEDAPSTDIYFPTDERSWVYSPLHYSARPASDGESDT

SEQ ID NO: 2 - protein, 668 aa, Homo sapiens
MELEVPDEAESAEAGAVPSEAAWAAESGAAAGLAQKKAAPTEVLSMTAQPGPGHGKKLGHRGVDASGETTYKKTTSSTLKGAIQLGIGYTVGHLSSKPERDVLMQDFYVVESIFFPSEGSNLTPAHHFQDFRFKTYAPVAFRYFRELFGIRPDDYLYSLCNEPLIELSNPGASGSLFYVTSDDEFIIKTVMHKEAEFLQKLLPGYYMNLNQNPRTLLPKFYGLYCVQSGGKNIRVVVMNNILPRVVKMHLKFDLKGSTYKRRASKKEKEKSFPTYKDLDFMQDMPEGLLLDADTFSALVKTLQRDCLVLESFKIMDYSLLLGVHNIDQHERERQAQGAQSTSDEKRPVGQKALYSTAMESIQGGAARGEAIESDDTMGGIPAVNGRGERLLLHIGIIDILQSYRFIKKLEHTWKALVHDGDTVSVHRPSFYAERFFKFMSNTVFRKNSSLKSSPSKKGRGGALLAVKPLGPTAAFSASQIPSEREEAQYDLRGARSYPTLEDEGRPDLLPCTPPSFEEATTASIATTLSSTSLSIPERSPSETSEQPRYRRRTQSSGQDGRPQEEPPAEEDLQQITVQVEPACSVEIVVPKEEDAGVEASPAGASAAVEVETASQASDEEGAPASQASDEEDAPATDIYFPTDERSWVYSPLHYSAQAPPASDGESDT

SEQ ID NO: 3 - protein, 688 aa, Rattus norvegicus
MELEVPDEAESAEAGAVTAEAAWSAESGAAAGMTQKKAILAEAPLVTGQPGPGHGKKLGHRGVDASGETTYKKTTSSTLKGAIQLGIGYTVGNLSSKPERDVLMQDFYVVESIFFPSEGSNLTPAHHFQDFRFKTYAPVAFRYFRELFGIRPDDYLYSLCNEPLIELSNPGASGSVFYVTSDDEFIIKTVMHKEAEFLQKLLPGYYMNLNQNPRTLLPKFYGLYCVQSGGKNIRVVVMNNVLPRVVKMHLKFDLKGSTYKRRASKKEKEKSLPTYKDLDFMQDMPEGLLLDSDTFGALVKTLQRDCLVLESFKIMDYSLLLGVHNIDQQERERQAEGAQSKADEKRPVAQKALYSTAMESIQGGAARGEAIETDDTMGGIPAVNGRGERLLLHIGIIDILQSYRFIKKLEHTWKALVHDGDTVSVHRPSFYAERFFKFMSSTVFRKSSSLKSSPSKKGRGALLAVKPLGPTAAFSASQIPSEREDVQYDLRGARSYPTLEDEGRPDLLPCTPPSFEEATTASIATTLSSTSLSIPERSPSDTSEQPRYRRRTQSSGQDGRPQEELHAEDLQKITVQVEPVCGVGVVVPKEQGAGVEVPPSGASAAATVEVDAASQASEPASQASDEEDAPSTDIYFFAHGRYWLFSPRRRRLRAVTPSHTGAPTDERSWVYSPLHYSARPASDGESDT

SEQ ID NO: 4 - protein, 4 aa, Artificial Sequence
  Synthetic consensus motif
  MISC_FEATURE (2)..(3): “Xaa” denotes any amino acid residue
  MISC_FEATURE (4)..(4): “Xaa” denotes a hydrophobic amino acid residue
YXXX

SEQ ID NO: 5 - protein, 27 aa, Artificial Sequence
  Synthetic consensus C-terminus
  MISC_FEATURE (16)..(16): “Xaa” denotes Arg or Gln
  MISC_FEATURE (17)..(18): “Xaa” denotes no amino acid residue or any amino acid residue
TDERSWVYSPLHYSAXXXPASDGESDT

SEQ ID NO: 6 - protein, 28 aa, Artificial Sequence
  Synthetic heptad repeat
KLEALEGRLDALEGKLEALEGKLDALEG

SEQ ID NO: 7 - protein, 11 aa, Homo sapiens
GSGSEAASLSS

SEQ ID NO: 8 - protein, 11 aa, Homo sapiens
GSGSTAGSLSS

SEQ ID NO: 9 - protein, 11 aa, Homo sapiens
GSGSDAASLSS

SEQ ID NO: 10 - protein, 11 aa, Homo sapiens
GSESIAESLSS

SEQ ID NO: 11 - protein, 4 aa, Homo sapiens
YSPL

SEQ ID NO: 12 - protein, 25 aa, Mus musculus
TDERSWVYSPLHYSARPASDGESDT

SEQ ID NO: 13 - protein, 16 aa, Artificial Sequence
  Synthetic peptide
CDERSWVYSPLHYSAR

SEQ ID NO: 14 - RNA, 21 nt, Artificial Sequence
  Synthetic oligonucleotide
aaguucuaugggcuguacugc

SEQ ID NO: 15 - RNA, 21 nt, Artificial Sequence
  Synthetic oligonucleotide
aaggaccuggacuucaugcag

SEQ ID NO: 16 - RNA, 19 nt, Artificial Sequence
  Synthetic oligonucleotide
gaaggcucuuguucacgau

SEQ ID NO: 17 - DNA, 32 bp, Artificial Sequence
  Synthetic oligonucleotide
ggagctgggtgtacttcccgcttcactatagc

SEQ ID NO: 18 - DNA, 35 bp, Artificial Sequence
  Synthetic oligonucleotide
ggagctgggtgtactccttccttcactatagcgcg

SEQ ID NO: 19 - DNA, 28 bp, Artificial Sequence
  Synthetic oligonucleotide
gggtgtactccccggttcactatagcgc

SEQ ID NO: 20 - DNA, 31 bp, Artificial Sequence
  Synthetic oligonucleotide
gctgggtgtactcccggcttcactatagcgc

SEQ ID NO: 21 - RNA, 21 nt, Artificial Sequence
  Synthetic oligonucleotide
aaguaccuguacuucaugcag

SEQ ID NO: 22 - RNA, 21 nt, Artificial Sequence
  Synthetic oligonucleotide
aagaaguuggagcacucuugg

(19) United States
(12) Patent Application Publication
(10) Pub. No.: US 2007/0161060 A1
(43) Pub. Date: Jul. 12, 2007

(54) Screening method for identifying an agent that modulates PIPKIgamma trafficking of E-cadherin

(76) Inventors: Richard A. Anderson, Cross Plains, WI (US); Xu dong Shi, Madison, WI (US); Shawn Bairstow, Gurnee, IL (US); Kun Ling, Madison, WI (US); Ari J. Firestone, Los Atlos, CA (US)

Correspondence Address: Jane Massey Licata;Licata & Tyrrell P.C., 66 E. Main Street, Marlton, NJ, 08053, US

(21) Appl. No.: 11/516,858

(22) Filed: Sep. 6, 2006

Related U.S. Application Data
(60) Provisional application No. 60/714,742, filed on Sep. 7, 2005.

(51) Int. Cl. G01N 33/574; A61K 38/55

(52) U.S. Cl. 435/7.23; 514/12

(57) ABSTRACT

The present disclosure teaches that PIPKIγ binds cadherins and adaptor protein μ-subunits and mediates trafficking of cadherins to and from the plasma membrane. Thus, the present invention relates to a method for identifying agents which modulate the trafficking or binding activities of a PIPKIγ. A method for diagnosing and prognosing a cancer derived from epithelial cells by detecting the amount and subcellular location of PIPKIγ is also provided as is a method for preventing or treating a disease or condition involving PIPKγ activity or cadherin localization.

This application claims benefit of U.S. Provisional Patent Application Ser. No. 60/714,742, filed Sep. 7, 2005, the content of which is incorporated herein by reference in its entirety.

This invention was made in the course of research sponsored by the National Institutes of Health (Grant Nos. 2 R01 GM57549-05 and 5 R01 GM51968-07). The U.S. government may have certain rights in this invention.

### BACKGROUND OF THE INVENTION

Development and maintenance of multicellular organisms requires precise and specific control of cell-cell adhesion (Gumbiner (1996) Cell 84:345-357). Cadherins are transmembrane receptors that regulate this cell-cell adhesion. In epithelial cells, E-cadherin and P-cadherin are required for adherens junction assembly. N-cadherin, expressed mainly in the brain and fibroblasts, mediates more dynamic cell-cell adhesions. The formation of adherens junctions is essential for morphogenesis, wound healing, and the retention of cell polarity and tissue integrity (Perez-Moreno, et al. (2003) Cell 112:535-548). In epithelia, adherens junctions are mediated by the calcium-dependent homophilic binding of E-cadherin molecules between neighboring cells (Gumbiner, et al. (1988) J. Cell Biol. 107:1575-1587; Pasdar and Nelson (1988) J. Cell Biol. 106:677-685; Pasdar and Nelson (1988) J. Cell Biol. 106:687-695). Inside the cell, β-catenin and then α-catenin anchor E-cadherin to the actin cytoskeleton, which plays a key role in assembly of adherens junctions (Gumbiner (1996) supra; Yap, et al. (1997) Annu. Rev. Cell Dev. Biol. 13:119-146). The function of the cadherin-catenin complex is modulated by dimerization, phosphorylation and ubiquitination (Yap, et al. (1997) supra). The amount of E-cadherin on the plasma membrane is controlled by precisely tuned exocytosis and endocytosis (Bryant and Stow (2004) Trends Cell Biol. 14:427-34). It has been suggested that p120-catenin (Davis, et al. (2003) J. Cell Biol. 163:525-534; Xiao, et al. (2003) J. Cell Biol. 163:535-545; Chen, et al. (2003) J. Cell Biol. 163:547-557), ARF6 (Palacios, et al. (2001) EMBO J. 20:4973-4986; Palacios, et al. (2002) Nat. Cell Biol. 4:929-936), tyrosine phosphorylation (Daniel and Reynolds (1997) Bioessays. 19:883-891), and the level of ubiquitionation (Fujita, et al. (2002) Nat. Cell Biol. 4:222-231; Murray, et al. (2004) Mol. Biol. Cell. 15:1591-1599) control the trafficking and assembly of E-cadherin in mammalian cells.

Epithelial cells give rise to the majority of cancers and E-cadherin is a powerful suppressor of carcinoma invasiveness. In many cancers, the loss or mis-targeting of E-cadherin is important for tumor invasiveness (Kang and Massague (2004) Cell 118:277-279; Hazan, et al. (2004) Ann. N. Y. Acad. Sci. 1014:155-163). The proteins β-catenin and p120-catenin directly associate with and modulate E-cadherin function. E-cadherin expression is regulated by β-catenin through activation of transcriptional suppressors Snail/Slug (Conacci-Sorrell, et al. (2003) J. Cell Biol. 163:847-857; Jamora, et al. (2003) Nature 422:317-322) while p120-catenin is important for modulating E-cadherin recycling (Davis, et al. (2003) supra; Xiao, et al. (2003) supra; Chen, et al. (2003) supra).

Phosphatidylinositol-4,5-biphosphate (PI4, 5P₂) regulates actin reorganization, focal adhesion assembly, and vesicular trafficking by modulating protein activities either via direct binding or indirectly through derived second messengers (Doughman, et al. (2003) J. Membr. Biol. 194:77-89). In mammalian cells, three isoforms of type I phosphatidylinositol phosphate kinase (PIPKI), α, β, and γ, are the major producers of PI4,5P₂ (Doughman, et al. (2003) J. Biol. Chem. 278:23036-23045).

Actin assembly in cells is regulated by a number of phosphoinositide-derived messengers (Janmey (1994) Annu. Rev. Physiol. 56:169-91; Janmey (1998) Physiol. Rev. 78:763-81; Janmey (1995) Chem. Biol.2:61-5; Lanier and Gertler (2000) Curr. Biol. 10:R655-7; Fawcett and Pawson (2000) Science 290:725-726). Many of the proteins modulated by PI4,5P₂ act to sever and nucleate F-actin, for example α-actinin, gelsolin and N-WASP. The other critical modulators of actin assembly are the small G-proteins Rac, Cdc42, Rho, and Arf (Hall (1998). Science 279:509-514; Schafer, et al. (2000) Traffic. 1:892-903; Moss and Vaughan (1998) J. Biol. Chem. 273:21431-21434; Ridley and Hall (1992) Cell 70:389-399; Ridley, et al. (1992) Cell 70:401-410; van Aelst and D'Souza-Schorey (1997) Genes Dev. 11:2295-2322; Chant and Stowers (1995) Cell 81:1-4; Nobes and Hall (1995) Cell 81:53-62; Symons (1995) Curr. Opin. Biotechnol. 6:668-674; Symons (1996) Trends Biochem. Sci. 21:178-181; Zigmond (1996) Curr. Opin. Cell Biol. 8:66-73). These G-proteins modulate the actin cytoskeleton in membrane ruffle assembly, the assembly of filopodia, lamellipodia, actin stress fibers, and focal adhesions. Phosphoinositide messengers regulate the activities of these G-proteins both directly and indirectly (Parker (1995) Curr. Biol. 5:577-9; Kam, et al. (2000) J. Biol. Chem. 275:9653-63; Brown, et al. (1998) Mol. Cell Biol. 18:7038-51; Donaldson and Jackson (2000) Curr. Opin. Cell Biol. 12:475-82). Membrane ruffling requires coordination between PIPKIα and Rac signaling (Doughman, et al. (2003) J. Biol. Chem. 278:23036-23045) and PIPKIγ661 is targeted to focal adhesions and modulates focal adhesion assembly via regulation by FAK, Src and talin (Ling, et al. (2002) Nature 420:89-93).

Integral membrane proteins are transported to and from the plasma membrane in lipid vesicles (Cremona and De Camilli (2001) J. Cell Sci. 114:1041-52; Kirchhausen (1999) Annu. Rev. Cell Dev. Biol. 15:705-32; Martin (2001) Curr. Opin. Cell Biol. 13:493-9; Takei and Haucke (2001) Trends Cell Biol. 11:385-91; Brett, et al. (2002) Structure (Camb) 10:797-809). This transport involves clathrin lattice assembly and is regulated by a family of adaptors known as the clathrin adaptor-protein complex (Kirchhausen (1999) supra). The adaptor-protein complexes consist of four distinct protein subunits. Of these subunits, the μ subunit contains the binding site for tyrosine or di-leucine motif sorting signals. The adaptor-protein complexes also contain many proteins that are regulated by PI4,5P₂ and indeed membrane traffic and endocytosis is a PI4,5P₂-dependent process (Cremona and De Camilli (2001) supra; Martin (2001) supra). Further, the μ-subunits of the AP1B/AP2 complex mediate endocytosis and sorting in epithelial cells (Gan, et al. (2002) Nat. Cell Biol. 4(8): 605-9; Folsch, et al. (1999) Cell 99:189-98), a process that is required for epithelial cell morphogenesis.

Spatial and temporal synthesis of PI4,5P₂ defines its function as a second messenger. PI4,5P₂ is maintained at relatively constant levels in cells (Anderson, et al. (1999) J. Biol. Chem. 274:9907-9910). Some agonists, such as growth factor stimulation or cell adhesion to the extracellular matrix, cause rapid but modest changes in cellular PI4, 5P₂ content (McNamee, et al. (1993) J. Cell Biol. 121:673-678; Chong, et al. (1994) Cell 79:507-513). Nevertheless, numerous cellular processes are regulated by PI4,5P₂ (McNamee, et al. (1993) supra; Toker (1998) Curr. Opin. Cell Biol. 10:254-61). It is thought that these processes are regulated by local changes in PI4,5P₂ synthesis, however, little is known as to how site-specific changes in PI4,5P₂ production are coordinated.

The C-termini of type I PIP kinase isoforms are sequence divergent, indicating that this region may be important for functional divergence. Each type I PIP kinase mRNA transcript is alternatively spliced resulting in multiple splice variants, each differentially localized for specific cellular functions (Ling, et al. (2002) supra; Anderson, et al. (1999) J. Biol. Chem. 274:9907-9910; Boronenkov and Anderson (1995) J. Biol. Chem. 270:2881-2884; Loijens and Anderson (1996) J. Biol. Chem. 271:32937-32943; Castellino, et al. (1997) J. Biol. Chem. 272:5861-5870; Ishihara, et al. (1996) J. Biol. Chem. 271:23611-23614; Ishihara, et al. (1998) J. Biol. Chem. 273:8741-8748; Itoh, et al. (1998) J. Biol. Chem. 273:20292-20299). Endogenous PIPKIA localizes both to the nucleus and membrane ruffles (Doughman, et al. (2003) supra; Boronenkov, et al. (1998) Mol. Biol. Cell 9:3547-3560) This isoform also functions in phagocytosis, which is a process dependent upon local changes in actin dynamics (Botelho, et al. (2000) J. Cell Biol. 151:1353-1368; Coppolino, et al. (2002) J. Biol. Chem. 277:43849-57). PIPKIα is regulated by Rac and is required for membrane ruffle assembly. Targeting to the plasma membrane is dependent not only upon Rac but also PDGF stimulus. Conversely, endogenous PIPKIβ localizes to the vesicular perinuclear region (Doughman, et al. (2003) supra)

The PIPKIγ isoform is alternatively spliced to form PIPKIγ635 and PIPKIγ661, which differ by a 26 amino acid C-terminal extension (Ling, et al. (2002) supra; Ishihara, et al. (1998) supra). The 26-amino acid C-terminus of PIPKIγ661 is sufficient for targeting PIPKIγ to focal adhesions (Ling, et al. (2002) supra). Focal adhesion-targeting is dependent upon an interaction between the PIPKIγ661 26-amino acid C-terminus and the talin FERM domain (Ling, et al. (2002) supra; Di Paolo, et al. (2002) Nature 420:85-89). This suggests that the Type I PIP kinase isoforms and splice variants are functionally diverse. The distinct targeting of these kinases dictates PI4,5P₂ production at specific sites throughout the cell, allowing for regulation of multiple cellular processes.

Assembly of focal adhesion protein complexes is initiated by the clustering of integrins binding to the extracellular matrix. Cultured cells form contacts with the matrix, which are important for signaling pathways that protect against apoptosis. In addition, cell adhesion is required for mitogen-stimulated cell growth in most cells and dynamic focal adhesion assembly is essential for cell migration in vitro (Critchley (2000) Curr. Opin. Cell Biol. 12:133-9; Burridge and Chrzanowska-Wodnicka (1996) Annu. Rev. Cell Dev. Biol. 12:463-519; Hemler and Rutishauser (2000) Curr. Opin. Cell Biol. 12:539-41; Giancotti (2000) Nat. Cell Biol. 2:E13-4; Giancotti and Ruoslahti (1999) Science 285:1028-32; Giancotti (1997) Curr. Opin. Cell Biol. 9: 691-700; Sanders, et al. (1998) Cancer Invest. 16:329-44; Turner (2000) Nat. Cell Biol. 2:E231-6).

Evidence suggests that PI4,5P₂ is critical for the assembly and functioning of focal adhesions (Gilmore and Burridge (1996) Nature 381:531-5; Martel, et al. (2001) J. Biol. Chem. 276:21217-21227). Upon binding PI4,5P₂, vinculin undergoes a conformational change that induces an association with other focal adhesion proteins (Martel, et al. (2001) supra), suggesting that PI4,5P₂ may be important in the assembly of the focal adhesion complex. In addition to vinculin, PI4,5P₂ regulates many actin-binding proteins, including profilin, gelsolin, α-actinin, and talin (Critchley (2000) supra; Burridge and Chrzanowska-Wodnicka (1996) supra; Hemler and Rutishauser (2000) supra; Giancotti (2000) supra; Giancotti and Ruoslahti (1999) supra; Giancotti (1997) supra; Sanders, et al. (1998) supra; Turner (2000) supra; Gilmore and Burridge (1996) supra; Martel, et al. (2001) supra).

Talin has been shown to specifically associate with PI4,5P₂ (Martel, et al. (2001) supra). The PI4,5P₂ association induces a conformational change in talin that enhances its interaction with β-integrins by exposing the integrin-binding site on talin. It has been suggested that PI4,5P₂-dependent signaling modulates assembly of focal adhesions by regulating integrin-talin complexes. These results demonstrate that activation of integrin binding to talin requires not only integrin engagement to the extracellular matrix, but also the binding of PI4,5P₂ to talin. This suggests a role for PI4,5P₂ generation in organizing the sequential assembly of focal adhesion components. Furthermore, masking of PI4,5P₂ by a specific pleckstrin homology domain confirms that PI4,5P₂ is necessary for proper membrane localization of talin and that this localization is essential for the maintenance of focal adhesions. Results consistent with this show that microinjection of antibodies against PI4,5P₂ inhibits assembly of stress fibers and focal adhesions (Burridge and Chrzanowska-Wodnicka (1996) supra; Gilmore and Burridge (1996) supra; Lauffenburger and Wells (2001) Nat. Cell Biol. 3:E110-2).

### SUMMARY OF THE INVENTION

The present invention is a method for identifying an agent that modulates the trafficking or binding activity of a PIPKIγ. The method involves contacting a PIPKIγ with a test agent in the presence of a cadherin or a μ-subunit and determining whether the agent modulates trafficking of the PIPKIγ or the cadherin, or binding of the PIPKIγ with the cadherin or the p-subunit, as compared to a control. A change in the trafficking or binding as compared to the control is indicative of the agent modulating the trafficking or binding activity of a PIPKIγ.

The present invention is also a method for diagnosing or prognosing a cancer by determining the amount and subcellular location of the PIPKIγ. This method involves contacting a biological sample with an agent which specifically binds a PIPKIγ; determining the amount and subcellular location of the PIPKIγ; and comparing the amount and subcellular location of PIPKIγ in the biological sample to the amount and subcellular location of PIPKIγ in a reference sample. The amount of membrane-localized PIPKIγ in the biological sample as compared to the reference sample is a diagnostic or prognostic indication of cancer.

The present invention further embraces a method for preventing or treating a disease or condition involving PIPKγ activity or cadherin localization. This method of the invention involves administering to a subject having or at risk of having a disease or condition involving PIPKγ activity or cadherin localization an effective amount of an agent which modulates the expression or trafficking or binding activity of a PIPKIγ thereby preventing or treating the disease or condition.

### BRIEF DESCRIPTION OF THE DRAWINGS

FIG. 1 depicts a schematic of the interactions between PIPKIγ and key effectors of epithelial/mesenchymal transition (EMT).

FIG. 2 depicts the AP2 interaction with the membrane, PI4,5P₂, and cargo protein. The μ-subunit is positioned to interact with the PIPKIγ when AP is bound to membrane or in the ‘open’ conformation.

FIG. 3 is a Kaplan Meier Survival plot for PIPKIγ higher expressors and low expresser breast tumors.

FIG. 4 depicts a comparison between human (SEQ ID NO:1), mouse (SEQ ID NO:2) and rat (SEQ ID NO:3) PIPKIγ amino acid sequences and a concensus thereof. C-termini of the PIPKIγ661 and PIPKIγ635 splice variants are shown (arrows) as is the Tyr-Xaa-Xaa-φ (SEQ ID NO:4) motif (boxed) and C-terminal amino acid residues of PIPKIγ661 (SEQ ID NO:5).

### DETAILED DESCRIPTION OF THE INVENTION

The epithelial/mesenchymal transition (EMT) is a hallmark of cancers derived from epithelial cells, such as breast, colon, ovarian and prostate cancers. More than two-thirds of all cancers are derived from epithelial cells (Hanahan and Weinberg (2000) supra). The loss of E-cadherin cell-cell contacts in epithelial cells leads to a loss of polarization and is a key indicator of transition from the epithelial to the mesenchymal phenotype (Arias (2001) supra; Savagner (2001) supra; Frame, et al. (2002) supra; Van Aelst and Symons (2002) supra; Thiery (2002) supra; Frisch (1997) supra; Birchmeier and Birchmeier (1995) supra; Hanahan and Weinberg (2000) supra). Clinically, the loss of cell surface E-cadherin is a prognosticator of poor patient survival. The loss of E-cadherin from cell-cell contacts is initiated by signaling pathways involving Src, Ras, and growth factors. It has been shown that endocytosis of E-cadherin is stimulated via ubiquitination by Hakai, an E3-ubiquitin-ligase related to Cbl that binds E-cadherin in a tyrosine phosphorylation-dependent manner (Fujita, et al. (2002) Nat. Cell Biol. 4:222-31).

It has now been shown that, in epithelial cells, endogenous PIPKIγ targets to adherens junctions by direct association with E-cadherin, and regulates trafficking of E-cadherin to and from the plasma membrane. Unexpectedly, a loss of PIPKIγ is highly correlative with E-cadherin expression or loss of membrane targeting in cancers such as breast carcinomas.

Epithelial cells polarize into apical and basolateral domains with E-cadherin located in the basolateral membrane. Endogenous PIPKIγ colocalized with E-cadherin at the basolateral membrane in polarized epithelial cells, but not with occludin. This was confirmed by constructing vertical sections of Z-series images. PIPKIγ is also present in a cytosolic vesicular compartment along with trace amounts of E-cadherin. The co-localization of PIPKIγ and E-cadherin indicated an interaction between PIPKIγ and a component of the adherens junction complex. Accordingly, E-cadherin was immunoprecipitated and PIPKIγ was found to be retained in these immunoprecipitates along with other cadherin-associated proteins. Immunoprecipitation with anti-PIPKIγ antibody confirmed that PIPKIγ also pulled down E-cadherin and other adherens junction components. These data demonstrate that PIPKIγ associates with adherens junction complexes. N-cadherin and VE-cadherin also associate with PIPKIγ, indicating an association with the classical cadherins.

Two splice variants of PIPKIγ, PIPKIγ635 and PIPKIγ661, were HA-tagged, expressed in HEK 293 cells, and their association with N-cadherin was analyzed. PIPKIγ635 and PIPKIγ661 bind N-cadherin indistinguishably, indicating that the association of PIPKIγ with cadherin does not depend upon the C-terminal 26 amino acids. However, the endogenous PIPKIγ associated with E-cadherin was the same size as PIPKIγ661. The association of PIPKIγ with classical cadherins indicates that this association may be a direct interaction between PIPKIγ and the cadherins. To address this, in vitro GST pull-down assays were performed using recombinant GST-tagged PIPKIγ and His-tagged cytoplasmic tail of E-cadherin. The E-cadherin cytoplasmic tail showed direct but low affinity binding to both GST-PIPKIγ635 and GST-PIPKIγ661, but not to GST alone or GST-PIPKIα, used as control.

Since E-cadherin molecules form lateral homodimers in vivo and oligomer formation is critical for adherens junction assembly and stability (Nagar, et al. (1996) Nature 380:360-364; Takeda, et al. (1999) Nat. Struct. Biol. 6:310-312; Patel, et al. (2003) Curr. Opin. Struct. Biol. 13:690-698), it was determined whether an E-cadherin cytoplasmic tail dimer preferentially associated with PIPKIγ. To construct a dimeric E-cadherin cytoplasmic tail, a heptad repeat sequence (Lys-Leu-Glu-Ala-Leu-Glu-Gly-Arg-Leu-Asp-Ala-Leu-Glu-Gly-Lys-Leu-Glu-Ala-Leu-Glu-Gly-Lys-leu-Asp-Ala-Leu-Glu-Gly; SEQ ID NO:6), which forms an a-helix, was inserted between the His-tag and the E-cadherin tail sequence to induce parallel dimer formation (Pfaff, et al. (1998) J. Biol. Chem. 273:6104-6109). When this construct was used in pull-down assays, it showed greater GST-PIPKIγ binding compared to the monomeric tail. This enhanced binding was not due to the heptad repeat tag since the heptad repeat-fused integrin cytoplasmic domain did not bind PIPKIγ. Additionally, when expressed in HEK293 cells, the c-Myc-tagged dimeric E-cadherin C-terminus (Myc-HR-ECDT) bound PIPKIγ and p120-catenin with ˜10-fold greater affinity compared to the monomer. The β-catenin, however, bound monomer and dimer with the same affinity, consistent with previous reports (Huber and Weis (1999) Cell 105:391-402). When expressed in MDCK cells, the dimer more efficiently triggered disassembly of adherens junctions. These data demonstrate that PIPKIγ directly binds to E-cadherin and preferentially binds the dimerized tail.

To determine if PIPKIγ binding to E-cadherin involves other adherens junction components, full-length wild-type or cytoplasmic tail-mutated E-cadherin constructs were expressed in HEK293 cells and assessed for endogenous PIPKIγ association. Endogenous PIPKIγ was immunoprecipitated and the associated E-cadherin was analyzed using an antibody specific for E-cadherin ectodomain. Elimination of either the p120-catenin (ECDΔp120, ⁷⁶²EED⁷⁶⁴ to AAA) or β-catenin (ECDΔβctn, ECD847, deletion of the last 35 amino acids) binding sites had no effect on PIPKIγ association. A chimera of truncated E-cadherin (deletion of the last 70 amino acids) fused to a truncated α-catenin that lacks the β-catenin binding site (Imamura, et al. (1999) J. Cell Biol. 144:1311-1322) abrogated both β-catenin and PIPKIγ binding. These results indicate that PIPKIγ binding to E-cadherin is independent of α-, β-, or p120-catenin and narrowed the PIPKIγ interaction region on E-cadherin to between residues 837 and 847. To refine the putative PIPKIγ binding site, the last 45 amino acids of E-cadherin was truncated (ECD836) and assayed for binding. The ECD836 truncation mutant lacked both β-catenin and PIPKIγ binding. The combined data demonstrates that PIPKIγ directly interacts with a highly conserved region including amino acids 837-847 of E-cadherin (Gly-Ser-Gly-Ser-Glu-Ala-Ala-Ser-Leu-Ser-Ser; SEQ ID NO:7), N-cadherin (Gly-Ser-Gly-Ser-Thr-Ala-Gly-Ser-Leu-Ser-Ser; SEQ ID NO:8), P-cadherin (Gly-Ser-Gly-Ser-Asp-Ala-Ala-Ser-Leu-Ser-Ser; SEQ ID NO:9) and VE-cadherin (Gly-Ser-Glu-Ser-Ile-Ala-Glu-Ser-Leu-Ser-Ser; SEQ ID NO:10).

To examine whether PIPKIγ could modulate E-cadherin function through the direct interaction, Myc-HR-ECDT was introduced into MDCK cells to compete with endogenous E-cadherin for PIPKIγ binding. PIPKIγ was trapped in the cytosol and adherens junctions identified by E-cadherin staining were lost in cells overexpressing Myc-HR-ECDT. To define the specificity of PIPKIγ regulation, the binding sites for p120 and β-catenin were deleted from Myc-HR-ECDT. When expressed, this construct disrupted the basolateral membrane targeting of E-cadherin and PIPKIγ, indicating that specificity for PIPKIγ binding was required. Further, overexpression of PIPKIγ was sufficient to fully rescue the loss of adherens junctions induced by Myc-HR-ECDT expression. These data establish that the specific interaction between PIPKIγ and E-cadherin plays a key role in E-cadherin function and appears to be a limiting factor in E-cadherin-mediated adherens junctions formation. These data are also supported by an E-cadherin germline mutation disclosed herein that eliminates PIPKIγ binding and its ability to form adherens junctions.

To define the functional role of PIPKIγ at adherens junctions, the endogenous expression of PIPKIγ was reduced using small interfering RNAs (siRNAs). Although cellular E-cadherin content was not changed, loss of PIPKIγ protein caused a loss of plasma membrane E-cadherin and its accumulation in a cytoplasmic compartment. The E-cadherin fluorescence intensity ratio (plasma membrane:cytosol) decreased from over 10-fold to less than one-eighth. The scrambled control siRNA had no effect on protein levels or targeting of PIPKIγ or E-cadherin. Upon loss of plasma membrane E-cadherin, the cell morphology underwent a dramatic transition from an epithelial to a mesenchymal phenotype. The E-cadherin protein level in PIPKIγ knockdown cells was not changed compared to wild-type cells. The mesenchymal phenotype indicates that PIPKIγ is required for E-cadherin-mediated adherens junctions assembly.

To further demonstrate this, MDCK stable cell lines where generated which expressed HA-tagged wild-type (PIPKIγ661WT) or kinase dead PIPKIγ661 (PIPKIγ661KD) the major endogenous PIPKIγ isoform associated with cadherin. Expression of PIPKIγ661WT or PIPKIγ661KD was induced by removing doxycyclin from the growth media. Cells were plated on coverslips and allowed to spread overnight. The cellular distribution of E-cadherin was then visualized by indirect immunofluorescence. E-cadherin staining was more intense at adherens junctions in the cells expressing PIPKIγ661WT compared to parental uninduced cells. The PIPKIγ661WT expressing cells also displayed a cytoplasmic compartment containing both PIPKIγ and E-cadherin. Conversely, upon PIPKIγ661KD expression, E-cadherin no longer targeted to adherens junctions and accumulated in the cytosol 16 hours post-plating of cells on the coverslips. The loss of E-cadherin from the plasma membrane correlated with increased PIPKIγ661KD expression in cells. Cells expressing PIPKIγ661KD formed E-cadherin-mediated cell-cell contacts when maintained at confluence for longer times than 72 hours. PIPKIγ661KD expression resulted in much slower adherens junction assembly, consistent with a dominant-negative effect for PIPKIγ661KD and also established a role for PI4,5P₂ generation in adherens junction assembly. The PI4,5P₂ requirement was reinforced by the observation that expression of PIPKIγ661WT facilitated assembly of E-cadherin and adherens junctions.

PI4,5P₂ is a signaling molecule that regulates multiple events, including vesicular trafficking and actin reorganization, which could affect E-cadherin assembly and adherens junction formation. Thus, changes in PIPKIγ expression levels may alter global PI4,5P₂ levels and induce non-specific cellular responses. Accordingly, the cellular PI4,5P₂ level was quantified by HPLC analysis and PI4,5P₂ distribution was assessed using GFP-PH_{PLCδ} following expression or knockdown of PIPKIγ. The data showed that there was no change in the global cellular PI4,5P₂ level when PIPKIγ content or activity was altered. Additionally, the overall structure of actin cytoskeleton showed no significant change between PIPKIγ661WT or KD overexpression cells and control cells. However, with depletion of PIPKIγ by siRNA treatment, cells exhibited an increase in actin stress fibers and prominent membrane ruffles, indicating a morphological transition from the polarized epithelial to migratory phenotype. This observation is consistent with the loss of E-cadherin-mediated adherens junctions, which play a key role in actin organization in polarized epithelial cells.

E-cadherin is modulated by both expression level and trafficking to and from the plasma membrane via exocytosis and endocytosis. E-cadherin-mediated adherens junction assembly depends upon extracellular calcium (Chitaev and Troyanovsky (1998) J. Cell Biol. 142:837 846). Depletion of extracellular calcium by EGTA results in a loss of E-cadherin homoligation, internalization of E-cadherin, disassembly of adherens junctions and scattering of the cells (Chitaev and Troyanovsky (1998) J. Cell Biol. 142:837-846). To analyze PIPKIγ modulation of E-cadherin trafficking, the exocytic and endocytic trafficking of E-cadherin was quantified in parental, PIPKIγ661WT, and PIPKIγ661KD expressing MDCK cells.

To quantify internalization, cell surface E-cadherin was biotinylated followed by calcium chelation with EGTA to induce E-cadherin internalization. PIPKIγ661WT expression considerably enhanced, whereas PIPKIγ661KD expression inhibited, E-cadherin internalization as compared to parental cells (Table 1). These data indicate that PIPKIγ plays a role in E-cadherin endocytosis, which is dependent upon PI4,5P₂ generation.

TABLE 1

| | Internalized Biotinylated E-Cadherin | | |
|---|---|---|---|
| | (% of Original | | |
| Time | Biotinylated E-Cadherin ± S.D.) | | |
| (Minutes) | Parental | PIPKIγ661WT | PIPKIγ661KD |
| 15 | 15± | 30± | 12± |
| 30 | 21± | 44± | 13± |
| 60 | 29± | 50± | 14± |
| 90 | 35± | 65± | 20± |
| 120 | 40± | 72± | 31± |

E-cadherin internalization can be reversed upon replenishment of calcium, providing a method to assess the role of PIPKIγ in recycling E-cadherin back to the plasma membrane. When cells were incubated in normal calcium containing medium following an EGTA treatment, E-cadherin was delivered to cell surface. The plasma membrane targeting of E-cadherin was accelerated when PIPKIγ661WT was expressed, whereas expression of PIPKIγ661KD blocked plasma membrane deposition of E-cadherin as compared to parental cells (Table 2).

TABLE 2

| | E-Cadherin on | | |
|---|---|---|---|
| Calcium | the Plasma Membrane | | |
| Rescue Time | (Fold of Control ± S.D.) | | |
| (Minutes) | Parental | PIPKIγWT | PIPKIγKD |
| 5 | 3.0± | 1.0± | 1.0± |
| 15 | 4.5± | 2.1± | 1.3± |
| 30 | 6.2± | 3.7± | 1.4± |
| 45 | 10.0± | 4.0± | 1.4± |
| 60 | 12.0± | 4.1± | 2.1± |

Additionally, in cells overexpressing PIPKIγ661WT, EGTA-induced E-cadherin internalization was much more rapid and appeared co-localized with PIPKIγ in an intracellular compartment, which has been reported as a syntaxin-4-positive basolateral recycling compartment (Ivanov, et al. (2004) Mol. Biol. Cell. 15:176-188). In contrast, overexpression of PIPKIγ661KD showed E-cadherin internalization as compared to parental controls.

To visually assess the plasma membrane targeting and assembly of newly synthesized E-cadherin, bead adhesion assays were performed using latex beads coated with recombinant soluble E-cadherin ectodomain (hE/Fc) (Yap, et al. (1997) Annu. Rev. Cell Dev. Biol. 13:119-146). To avoid non-specific signal resulting from indirect immunofluorescence, GFP or DsRed fusion proteins were employed. Eight to ten hours after ttansfection, the beads were added to MCF10A cells and allowed to bind for 20 minutes. Newly synthesized GFP-E-cadherin was assembled on the surface of the hE/Fc-coated beads where DsRed-PIPKIγ661 was visualized. In addition, GFP-fused PH domain of PLCδ colocalized with the hE/Fc-coated beads, indicating generation of PI4,5P₂ on the bead surface. When PIPKIγ661KD was expressed in these cells, both the enrichment of PI4,5P₂ and assembly of E-cadherin on the surface of the beads were blocked. These combined results demonstrate that PIPKIγ661 and the PI4,5P₂ generated modulate E-cadherin deposition into the plasma membrane and adherens junction assembly.

The assembly of adherens junctions is a complex process requiring E-cadherin targeting to the plasma membrane and formation of extracellular anti-parallel adhesive dimers between cells. E-cadherin trafficking to and from the plasma membrane is a key event in adherens junction assembly and involves the vesicular-trafficking machinery. The results provided herein reveal that PIPKIγ is a new member of the adherens junction protein complex, directly binding E-cadherin and regulating E-cadherin trafficking, including both exocytosis and endocytosis. Evidence suggests that PI4,5P₂ has multiple roles in vesicular trafficking. For example, PI4,5P₂ and PIPKIγ play a role in the regulation of synaptic vesicle trafficking (Di Paolo, et al. (2004) Nature. 431:415-422). Several components of vesicular trafficking machinery are modulated by binding to PI4,5P₂ (Cullen, et al. (2001) Curr. Biol. 11:R882-893; Martin (2001) Curr. Opin. Cell Biol. 13:493-499; Simonsen, et al. (2001) Curr. Opin. Cell Biol. 13:485-492), such as adaptor protein complexes, AP180, epsin, and kinesin (Klopfenstein, et al. (2002) Cell 109:347-358). The basolateral sorting of E-cadherin occurs via a dileucine motif in the juxtamembrane region (Miranda, et al. (2001) J. Biol. Chem. 276:22565-22572), which is recognized by a clathrin adaptor complex, such as AP-1 (Rapoport, et al. (1998) EMBO J. 17:2148-2155). In epithelial cells, AP-lB is specifically expressed and mediates basolateral trafficking (Gan, et al. (2002) Nat. Cell Biol. 4:605-609).

In a yeast 2-hybrid screen using the C-terminal 200 amino acid domain of PIPKIγ661 as bait, it was found the fragments of both μ1β (amino acid residues 135-423) and μ2 subunits (full length) of adaptor protein complexes interact with PIPKIγ661. This observation was of interest because the μ-subunits are key regulatory subunits of the AP complexes (Bonifacino and Traub (2003) Annu. Rev. Biochem. 72:395-447). AP1B is required for basolateral membrane recycling in epithelial cells (Gan, et al. (2002) Nat. Cell Biol. 4:605-609) and AP2 is an important component of clathrin-dependent endocytosis machinery. Both complexes are regulated by PI4,5P2 (Martin (2001) Curr. Opin. Cell Biol. 13:493-499). The PIPKIγ interaction with μ1β was confirmed by direct binding of purified components, and the association between PIPKIγ and AP1 was established by co-immunoprecipitation of endogenous proteins. Further, PIPKIγ635 did not interact with either μ-subunit, indicating that the final C-terminal 26 amino acid residues of PIPKIγ661 are required. The observation that the PIPKIy-positive vesicle-like structures significantly co-localized with α-adaptin, another subunit of AP1, reinforces this association. Additionally, E-cadherin and PIPKIγ were both partially localized in γ-adaptin in cytoplasmic compartments after removal of calcium, indicating a functional link among E-cadherin, PIPKIγ 661 and AP1 in E-cadherin recycling. In addition, binding of μ1β-adaptin stimulated the kinase activity of PIPKIγ 661, whereas binding of the soluble E-cadherin cytoplasmic domain (His-HR-ECDT) had no effect on PIPKIγ 661 activity under these conditions.

Endogenous E-cadherin forms a protein complex with PIPKIγ and AP1. The association of E-cadherin with PIPKIγ 661 and AP1 was disrupted by depletion of calcium which causes the disassembly of adherens junctions and internalization of E-cadherin. When calcium was restored and E-cadherin recycling was triggered, E-cadherin was re-assembled into the PIPKIγ 661-AP1 complex. To further examine the interactions between E-cadherin, PIPKIγ661 and AP1, a GST pull-down analysis was performed using purified GST-μ1β, His-PIPKIγ661, and His-HR-ECDT. The results of this analysis indicated that there was no direct interaction between μ1β and E-cadherin cytoplasmic domain. However, PIPKIγ661 was able to link E-cadherin C-terminus (HR-ECDT) to μ1β as shown by combined pulled down by GST-μ1β but only in the presence of PIPKIγ661. The PIPKIγ661 C-terminal contains a Tyr-Xaa-Xaa-φ (SEQ ID NO:4) cargo-binding motif (Bonifacino and Traub (2003) Annu. Rev. Biochem. 72:295-447) in the final C-terminal 26 amino acid residues (Tyr-Ser-Pro-Leu; SEQ ID NO:11). The tyrosine in the Tyr-Xaa-Xaa-φ (SEQ ID NO:4) motif is required for high affinity binding to the μ-subunits, and when substituted with a phenylalanine the binding is reduced (Ohno, et al. (1998) J. Biol. Chem. 273:25925-25921). When using a PIPKIγ661 (Tyr644Phe) mutant, which showed diminished binding with μ1β, the amount of E-cadherin C-terminus pulled down by μ1β was reduced significantly, indicating that the interaction between PIPKIγ661 and the μ1β-subunit is necessary and sufficient to link E-cadherin to the AP1 complex. This scaffolding interaction is also required for transport of E-cadherin in vivo as disclosed herein.

Since AP complexes play an important role in protein transport, the data herein suggest that PIPKIγ661 regulates E-cadherin trafficking via direct interaction with and regulation of the AP complexes. Such a model would require APLB for E-cadherin transportation to the plasma membrane. To address this, LLC-PK1 cells, which do not express the μ1β subunit (i.e., are AP1B deficient) were employed. In LLC-PK1 cells, many basolateral proteins are mistargeted and cells do not polarize (Folsch, et al. (1999) Cell 99:189-198). To assess the role of μ1β-subunit in transport, GFP-E-cadherin was expressed. A small fraction of the GFP-E-cadherin was able to translocate to the plasma membrane, however, the majority was observed in a perinuclear vesicular compartment where endogenous PIPKIγ was recruited, indicating an inefficient post-trans-Golgi transportation. Upon rescue by expression of μ1B in the LLC-PK1:μ1β cells, GFP-E-cadherin was targeted to cell-cell adhesion sites efficiently and colocalized with PIPKIγ at these sites.

As PIPKIγ661 associates with AP1, PIPKIγ661 may recruit AP1 to E-cadherin containing membranes when recycling of E-cadherin is triggered. To determine this, confluent parental or PIPKIγexpressing MDCK cells were treated with EGTA for 30 minutes to induce E-cadherin endocytosis followed by replenishment of calcium for 10 minutes to induce exocytic trafficking. Cells were fixed and stained for AP1, E-cadherin, and PIPKIγ. In PIPKIγ661 overexpressing cells, E-cadherin was rapidly deposited in the basolateral membrane and AP1 was recruited to the basolateral membrane colocalizing with E-cadherin and PIPKIγ661. In parental cells, endogenous AP1 remained largely perinuclear with a small fraction targeting to the plasma membrane. PIPKIγ635 does not interact with AP1 and in cells overexpressing PIPKIγ635, the AP1 organization was strikingly different as concentrated in a central perinuclear compartment with no localization near the plasma membrane and little colocalization with E-cadherin, which was trapped in the cytosol. In PIPKIγ661KD expressing cells, AP1 weakly localized beneath the plasma membrane or showed strong co-localization with E-cadherin and PIPKIγ661KD in a large perinuclear compartment, but there was no detectable plasma membrane E-cadherin. These data indicate a direct interaction between AP1 with PIPKIγ661 (and its PIP kinase activity) which is necessary for recruitment of AP1 to the plasma membrane and efficient exocytic trafficking of E-cadherin in vivo.

If the association of PIPKIγ661 with AP complexes were required for E-cadherin trafficking, PIPKIγ635 which does not interact with AP complexes but E-cadherin would not support this function and may act as a dominant negative. In confluent PIPKIγ635 overexpressing MDCK cells, both endogenous E-cadherin and ectopically expressed GFP-fused E-cadherin were sequestered in a cytosolic compartment with a phenotype similar to that observed when endogenous PIPKIY was knocked down. Although PIPKIγ661 and PIPKIγ635 bind E-cadherin identically in vitro, PIPKIγ635 does not bind AP1 and could not substitute for PIPKIγ661 in regulation of E-cadherin trafficking in vivo. When internalization and recycling of E-cadherin was quantified by surface biotinylation, overexpression of PIPKIγ635 inhibited trafficking to and from the plasma membrane compared to parental cells. As PIPKIγ635 does not interact with AP complexes, these results demonstrate that E-cadherin trafficking requires a functional interaction between PIPKIγ661 and AP1B.

If PIPKIγ serves as an adaptor between E-cadherin and AP complexes, the interaction of PIPKIγ with E-cadherin would be crucial for PIPKIγ661 to fulfill this role. In this context, the E-cadherin mutant lacking or with diminished PIPKIγbinding should not be transported efficiently to the plasma membrane. A Val832Met germline mutation was identified in hereditary diffuse gastric cancer (Yabuta, et al. (2002) Int. J. Cancer 101:434-441), which lacks the ability to mediate cell-cell adhesion and to suppress invasion (Suriano, et al. (2003) Oncogene 22:5716-5719). In these patients, the wild-type E-cadherin gene is repressed, and only the mutant is expressed in the carcinomas (Yabuta, et al. (2002) supra). Of interest, the Val832Met mutation lies in PIPKIγ binding region. To determine whether this mutation impacts PIPKIγ binding, E-cadherin Val832Met was introduced into HEK293 cells and immunoprecipitation demonstrated that this mutant had a substantially lower ability to bind PIPKIγ. Consistent with published data (Suriano, et al. (2003) supra), β-catenin binding was normal. The basolateral transport of this Val832Met mutation was subsequently determined in both LLC-PK1:μ1β and MDCK cells using GFP-fused E-cadherin Val832Met. Although the Val to Met mutant was visualized on the plasma membrane as reported by others (Suriano, et al. (2003) supra), a large accumulation of this E-cadherin mutant was observed in a cytosolic compartment. This phenotype was similar to that of wild-type E-cadherin observed in the PIPKIγ635 overexpressing cells. Compared to the mutant, wild-type E-cadherin in LLC-PK1:μ1β and MDCK cells was transported efficiently to the basolateral membrane and little was visualized in the cytosol. This result is consistent with a requirement for an interaction between E-cadherin and PIPKIγ661 for normal trafficking of E-cadherin.

To further analyze the association of PIPKIγ661 with the μ2-subunit of AP2, a GST pulldown approach was used. Since the full-length μ2-subunit is primarily insoluble in Escherichia coli, a truncation mutant was generated. This was accomplished by deleting of the bulk of the N-terminal domain and replacing it with GST. The resulting soluble construct contained the complete linker domain and the C-terminal domain. The GST pulldown was then performed by incubating PIPKIγ661 with GST-μ2 in the presence of glutathione conjugated SEPHAROSE™ beads. The results of this analysis showed that PIPKIγ661 directly associated with GST-μ2, but did not associate with GST alone.

In vivo, AP2 serves as an adaptor for binding to several other proteins involved in clathrin-mediated endocytosis. Consequently, to insure that this interaction was relevant in vivo, endogenous AP2 was immunoprecipitated from HEK293 cells using a monoclonal antibody specific for α-adaptin. The precipitated complexes were washed extensively and resolved by SDS-PAGE. Using a polyclonal antibody, PIPKIγ was detected in the α-adaptin lane but not in the normal mouse IgG lane. The reciprocal experiment, using the monoclonal antibody specific for α-adaptin, yielded concurrent results. It was unexpected that only the highest molecular weight band was retained by immunoprecipitated AP2. This band corresponded to PIPKIγ661, as the PIPKIγ polyclonal antibody detected both PIPKIγ661 and PIPKIγ635 splice variants, as seen in the lysate lane.

Since PI4,5P₂ has extensively been shown to be an integral component of the endocytic process, it was determined if PIP Kinase activity was necessary for this interaction in vivo. Wild-type and kinase-inactive PIPKIγ661 (i.e., PIPKIγ661WT and PIPKIγ661KD, respectively) were transfected into HEK293 cells via calcium phosphate. AP2 was then immunoprecipitated using monoclonal antibodies specific for the α-subunit. The results of this analysis indicated that both PIPKIγ661WT and PIPKIγ661KD associated with AP2 in vivo. In addition, PIPKIγ661KD appears to associate with the AP2 complex with slightly higher affinity as compared to PIPKIγ661WT.

The endogenous coimmunoprecipitation data indicated that AP2 preferentially associates with the highest molecular weight species of PIPKIγ detected by the PIPKIγ polyclonal antibody. A coimmunoprecipitation approach was used to confirm which of the two splice variants could associate with AP2 in vivo. HEK293 cells were transfected with PIPKIγ661 or PIPKIγ635, and endogenous AP2 was immunoprecipitated with the α-adaptin-specific antibody. This experiment indicated that only PIPKIγ661 was capable of binding to AP2 in vivo. This result indicated that the in vivo binding site for μ2 was localized in the C-terminal 26 residues of PIPKIγ661. To narrow the specific binding site, three previously generated truncations of PIPKIγ661 were employed in the same coimmunoprecipitation approach (Ling, et al. (2003) J. Cell Biol. 163:1339-1349). Truncation at Trp642 resulted in reduction of associated PIPKIγ to background levels observed in the normal mouse IgG control.

Upon closer inspection of the sequence contained between the Trp642 and Tyr649 truncations, a tyrosine sorting motif (644Tyr-Ser-Pro-Leu647; SEQ ID NO:11) was identified. Several point mutations were generated in accordance with a peptide library screen known in the art (Ohno, et al. (1998) supra) with the intention of weakening PIPKIγ661 binding to AP2. A mutation intended to strengthen binding was also generated as a positive control. Each of these HA-tagged constructs was transfected into HEK293 cells and endogenous AP2 was immunoprecipitated with an α-adaptin-specific antibody. Associated PIPKIγ661 was then detected by immunoblot with an HA-specific monoclonal antibody.

Mutation of either Tyr644, Pro646 (Tyr+2) or Leu647 (Tyr+3) to the most disfavored residues resulted in disruption of the interaction. The Tyr+1 position was previously shown to have little contribution to binding affinity (Ohno, et al. (1998) supra), and mutation of Ser645 to the least favored residue, phenylalanine, had little effect on AP2 binding. Likewise, mutation of Pro646 (Tyr+3) to the most favored residue, arginine, did not alter PIPKIγ661 binding to AP2. These in vivo results were confirmed via GST pulldown experiments. GST-μ2 was used to pulldown PIPKIγ661, PIPKIγ635, PIPKIγTyr644Phe, or PIPKIγ Leu647Val. The incubation buffer was supplemented with 1% bovine serum albumin to inhibit non-specific interactions. PIPKIγ661 was specifically retained by GST-μ2, while none of the PIPKIγ constructs were associated with GST alone. These combined results indicate that PIPKIγ661 contains a tyrosine sorting motif which is recognized by the μ2-subunit of AP2 and mediates the direct interaction between these two proteins.

The prior art has also demonstrated that phosphorylation of the tyrosine residue within Tyr-Xaa-Xaa-φ (SEQ ID NO:4) sorting motifs disrupts the association of such motifs with the μ2-subunit (Ohno, et al. (1998) supra). Moreover, Tyr644 of PIPKIγ661 is also phosphorylated by Src in a Focal Adhesion Kinase-dependent manner (Ling, et al. (2002) Nature 420:89-93; Ling, et al. (2003) supra). Consequently, tyrosine phosphorylation of this residue might disrupt the association between PIPKIγ661 and the μ2-subunit. To address this, in vitro GST pulldown assay were performed by incubating GST-μ2 with PIPKIγ661 or tyrosine phosphorylated recombinant PIPKIγ661, generated using established methods (Bairstow, et al. (2005) J. Biol. Chem. 280:23884-23891). Tyrosine phosphorylated PIPKIγ661 associated with much lower affinity as compared to nonphosphorylated PIPKIγ661. This result is consistent with the requirement of an unphosphorylated tyrosine within the Tyr-Xaa-Xaa-φ (SEQ ID NO:4) sorting motif and also might serve as a regulatory mechanism for the interaction between AP2 and PIPKIγ661.

Since it was determined that PIPKIγ661 interacts with the AP2 complex both in vivo and in vitro, it was examined whether PIPKIγ shared a similar subcellular localization with AP2. Endogenous PIPKIγ and AP2 were immunostained with antibodies specific for PIPKIγ and α-adaptin, respectively, in MDCK cells and examined by confocal microscopy. PIPKIγ was primarily targeted to the plasma membrane and to sites of cell-cell contacts in MDCK cells. This localization also overlapped with the punctuate plasma membrane staining of endogenous AP2, and could be observed not only at the plasma membrane, but also at discrete locations within the cytoplasm. This partially overlapping staining at the plasma membrane was not unexpected, as PIPKIγ661 has been shown to serve several roles at the plasma membrane, including regulation of focal adhesion assembly via direct interaction with talin (Ling, et al. (2002) supra; Di Paolo, et al. (2002) Nature 420:85-92). It is of note that the PIPKIγ polyclonal antibody employed in this analysis detects multiple PIPKIγ splice variants expressed in MDCK cells, and it has been shown that the interaction with AP2 is specific for only the PIPKIγ661 splice variant.

To further examine the specificity of the interaction between PIPKIγ661 and AP2, HA-tagged PIPKIγ661WT, PIPKIγ661 Tyr644Phe and PIPKIγ661 Ser645Phe constructs were expressed in MDCK cells. Expressed under normal conditions, all three HA-PIPKIγ661 constructs were targeted to the plasma membrane in a similar manner. However, upon stimulation of clathrin-mediated endocytosis via treatment with transferrin, distinct colocalization patterns were observed. Both wild-type and Ser645Phe PIPKIγ661 colocalized with AP2 in internalized vesicular structures upon treatment with transferrin. However, in cells expressing PIPKIγ661 Tyr644Phe, the HA-PIPKIγ signal remained at the plasma membrane and was not significantly internalized under identical conditions. These data collectively support both the specificity of the PIPKIγ661/AP2 interaction demonstrated in vivo and in vitro and also reinforce the functional implications observed in transferrin uptake experiments described herein.

The interaction between PIPKIγ661 and AP2 may be a transient event, occurring primarily to facilitate targeting of PIPKIγ661 to sites of endocytosis for localized generation of PI4,5P₂. This dynamic association would be necessary for recognition of cargo proteins by the μ2-subunit upon assembly of the AP2 complex onto the plasma membrane, since PIPKIγ661 would occupy the cargo binding site when directly associated with AP2. Consequently, the vesicular cytoplasmic colocalization patterns observed in may be PIPKIγ661 directly associated with AP2 during the cycling of the endocytic machinery.

There is considerable evidence that endocytosis from the plasma membrane mediated by the AP2 complex is a PI4,5P₂-dependent process (Martin, et al. (2001) Curr. Opin. Cell Biol. 13:493-499; Cockcroft and De Matteis (2001) J. Memb. Biol. 180:187-194). The observed endogenous colocalization and the direct interaction between the μ-subunit of AP2 and PIPKIγ661 collectively indicate that this interaction may have implications on AP2 function. To address this possibility stable MDCK cell lines inducibly expressing either wild-type or kinase dead PIPKIγ661 were generated. PIPK expression was induced by withdrawal of doxycycline from the growth media. After 72 hours of expression, the cells were subjected to an endocytosis assay via treatment with ALEXA Fluor 647 transferrin and the amount of internalized transferrin was then assessed via flow cytometry. The results from these assays demonstrated that stable expression of wild-type PIPKIγ661 resulted in an over 40% average increase of mean fluorescence intensity relative to nonexpressing cells. The opposite effect was observed in cells expressing kinase dead PIPKIγ661, with a 25% average decrease of intensity.

To confirm that these effects on transferrin endocytosis were due to a specific interaction with PIPKIγ661, MDCK stable cell lines inducibly expressing wild-type or kinase dead PIPKIγ635 were also generated. Induced expression of either wild-type or kinase dead PIPKIγ635, under the same conditions, had no appreciable effect on transferrin endocytosis. In addition, similar results were also obtained using an MDCK stable cell line inducibly expressing PIPKIγ661 with a Tyr644Phe mutation. These results were consistent with both the in vitro and in vivo interaction data, which indicated that only the PIPKIγ661 splice variant containing the Tyr-Ser-Pro-Leu (SEQ ID NO:11) motif is capable of direct interaction with the μ-subunit of the AP2 complex.

The prior art has also proposed a link between trafficking of the AP2 complex and the focal adhesion protein talin (Morgan, et al. (2004) J. Cell Biol. 167:43-50). Additionally, the established binding site for talin on PIPKIγ661 does overlap with the Tyr-Ser-Pro-Leu (SEQ ID NO:11) motif necessary for the direct interaction with AP2 (Ling, et al. (2002) supra; Di Paolo, et al. (2002) supra). To uncouple these two distinct interactions for PIPKIγ661, a stable cell line expressing the Ser645Phe mutant was generated. This mutation does not affect binding to the AP2 complex in vivo; however, this mutation does disrupt the interaction between PIPKIγ661 and talin as observed by coimmunoprecipitation. This phenotype was further confirmed by indirect immunofluorescence. Expression of wild-type PIPKIγ661 resulted in a distinct colocalization pattern with talin at the plasma membrane in MDCK cells. Expression of PIPKIγ661 Ser645Phe, on the other hand, showed little colocalization with a more diffuse talin staining pattern, similar to that observed upon expression of PIPKIγ661 Tyr644Phe.

In subsequent endocytosis assays, expression of the Ser645Phe mutant also had a stimulatory effect in MDCK cells, but to a greater extent than that of wild-type PIPKIγ661. This higher response may simply be the consequence of a lack of competition with talin for binding to PIPKIγ661. Mutation of this residue might also inhibit Src-mediated phosphorylation of Tyr644. It has been shown that both phosphorylation and mutation of Ser645 results in diminished phosphorylation of Tyr644 (Lee, et al. (2005) J. Cell Biol. 168:789-799). Consequently, the Ser645Phe mutant might not be susceptible to the disruptive effect of Tyr644 phosphorylation observed in vitro.

Since potential changes in PI4,5P₂ levels alone might be attributed to the observed effects, total cellular PI4,5P₂ levels were quantified via metabolic labeling and HPLC. The PIPKIγ661WT and PIPKIγ661KD stable MDCK cell lines were cultured in the presence of ³H-myo-inositol and protein expression was induced for the same duration as in the transferrin uptake assays. The cellular lipids were extracted, deacylated and resolved by anion exchange. The PI4,5P₂ peak was identified using a deacylated PI4,5P₂ standard and quantified by total counts using an inline flow scintillation counter. Induction of expression was verified by immunoblot of unlabeled cells. The results of this analysis showed that the expression of either PIPKIγ661WT or PIPKIγ661KD did not have a significant effect on the total cellular PI4,5P₂ level. Since the total PI4,5P₂ levels were not significantly affected by increased PIPKIγ661WT or PIPKIγ661KD expression, it is likely a modulation of highly localized pools of PI4,5P₂ that is responsible for the observed effects on transferrin endocytosis. The relatively stable level of cellular PI4,5P₂ is also consistent with previously reported observations for increased expression of PIPKIγ661 in other cell lines (Alonso, et al. (2004) Cell 117:699-711).

An RNAi based approach was also employed as an alterative method for addressing a possible role for PIPKIγ661 in transferrin receptor endocytosis. Using an siRNA oligo specific for the human PIPKIγ, PIPKIγ expression was reduced in HeLa cells. These cells were then used in the same transferrin uptake assay as utilized with the MDCK PIPKIγ661 stable cell lines. A non-specific siRNA oligo was used as a control for normalizing transferrin uptake. As an additional control, PIPKIα levels were also reduced with an siRNA oligo specific for this isoform. These results showed that transferrin uptake was inhibited on average by 50% in cells transfected with PIPKIγ siRNA. However, no significant effect was observed with either nonspecific control siRNA or with siRNA specific for PIPKIα. The results observed for knockdown of PIPKIα via siRNA was also consistent with results reported previously in HeLa cells (Alonso, et al. (2004) supra).

A change in the expression level of the transferrin receptor could also contribute to the observed inhibition of transferrin endocytosis upon knockdown of PIPKIγ. To rule out this possibility, the transferrin receptor expression level was assessed under these conditions by western blot analysis. Knockdown of PIPKIγ661 did not affect the overall expression level of the transferrin receptor, indicating that the observed effects on endocytosis were a direct consequence of reduced expression of PIPKIγ.

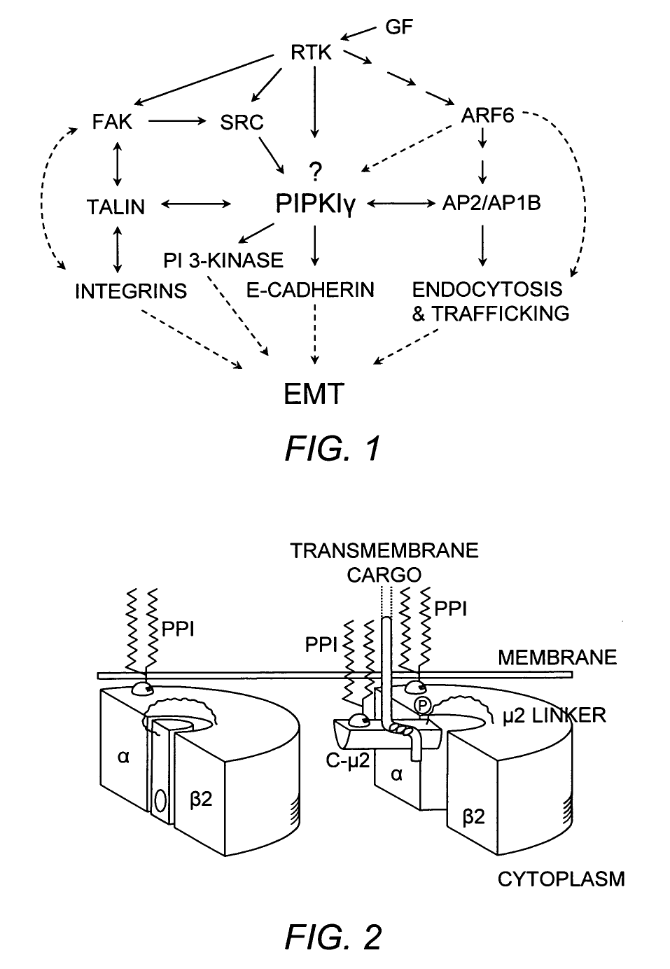

The results disclosed herein reveal a novel mechanism where PIPKIγ661 functions as both a scaffolding and a signaling molecule to regulate E-cadherin trafficking (FIG. 1). This occurs via the bridging interaction of PIPKIγ661 with E-cadherin and AP complexes. This dual interaction supports a mechanism for highly regulated generation of PI4,5P₂ that spatially drives the assembly of the trafficking machinery and specifically controls E-cadherin trafficking. PIPKIγ661 associates with both AP2 and AP1 B via a direct interaction with their μ-subunits. This interaction occurs via the Tyr-Ser-Pro-Leu (SEQ ID NO:11) motif of PIPKIγ661, which has been confirmed to be a Tyr-Xaa-Xaa-φ (SEQ ID NO:4) sorting motif recognized by μ-subunits. It has now been shown that mutation of any of the key residues within this motif results in disruption the interaction with the μ2-subunit both in vivo and in vitro. The direct interaction between AP2 and PIPKIγ661 provides a mechanism for targeting PIPKIγ661 to sites of endocytosis at the plasma membrane. Consequently, this would result in generation of a highly concentrated pool of PI4,5P₂ at these sites.

The structure for the AP2 core provides some insight to a possible mechanism for regulation of this interaction. In the AP2 crystal, the μ2-subunit is buried in a grove formed by the α- and β2-subunits (Collins, et al. (2002) Cell 109:523-535). In this ‘closed’ conformation, the μ2-subunit Tyr-Xaa-Xaa-φ (SEQ ID NO:4) docking site is positioned away from the membrane docking site of the complex (FIG. 2). It has been proposed that phosphorylation of the linker domain of μ2 might trigger a conformational change that would allow the subunit to swing out of the pocket into an ‘open’ conformation and bind to cargo motifs at the plasma membrane (Collins, et al. (2002) supra; Conner, et al. (2003) Traffic 4:885-890; Conner and Schmid (2002) J. Cell Biol. 145:921-929; Ricotta, et al. (2002) J. Cell Biol. 156:791-795). This structural shift allows for enhanced AP2 membrane association via a direct interaction between μ2 and PI4,5P₂, which is not possible in the nonphosphorylated, ‘closed’ conformation (Honing, et al. (2005) Mol. Cell 18:519-531). Since PIPKIγ661 binding would occupy the cargo binding site, PIPKIγ661 could bind to AP2 in this inactive conformation.

It is believed that upon docking to the plasma membrane, PIPKIγ661 would be displaced from μ2 by either a conformational change in μ2 or by competition of sorting motifs of higher affinity. This displacement would also be facilitated by tyrosine phosphorylation of PIPKIγ661. It has been demonstrated that phosphorylation of the tyrosine within the Tyr-Xaa-Xaa-φ (SEQ ID NO:4) motif inhibits the interaction with μ2 in cargo peptide binding studies (Ohno, et al. (1996) J. Biol. Chem. 271:29009-29015). Moreover, it has been demonstrated that Tyr644 of PIPKIγ661 is preferentially phosphorylated by Src (Ling, et al. (2003) supra) and tyrosine phosphorylation of PIPKIγ661 disrupts the association with the μ2-subunit in vitro. Additionally, several tyrosine kinase receptors trigger Src activation upon binding to extracellular ligands (Thomas and Brugge (1997) Annu. Rev. Cell Dev. Biol. 13:513-609). Consequently, PIPKIγ661 would likely become phosphorylated and dissociate from AP2 upon targeting to activated tyrosine kinase receptors or to sites where Src may be active. Therefore, phosphorylation of Tyr644 on PIPKIγ661 could serve as an important regulatory mechanism for the interaction between PIPKIγ661 and AP2 at the plasma membrane.

A dileucine motif in the juxtamembrane region of E-cadherin cytoplasmic domain is required for basolateral sorting (Miranda, et al. (2001) J. Biol. Chem. 276:22565-22572), and this motif has been proposed to be a cargo signal recognized by the β-subunit of the AP1 complex (Rapoport, et al. (1998) EMBO J. 17:2148-2155). PIPKIγ661 recruits AP1B to E-cadherin via its interaction with μ1β and this interaction could be further stabilized via the interaction of the E-cadherin dileucine motif with the β-subunit of AP1. These combined interactions serve as a specific signal for exocytic targeting and basolateral sorting. Although Rab11 was shown to be required for E-cadherin transportation from the trans-Golgi network to the plasma membrane in HeLa cells (Lock and Stow (2005) Mol. Biol. Cell. 16:1744-1755), none of the E-cadherin accumulating intracellular compartments observed herein, when PIPKIγ661 or AP1B function was disrupted, showed colocalization with endogenous Rab11. This is most likely due to the multiple pathways for E-cadherin trafficking preferentially utilized by different types of cell (Bryant and Stow (2005) supra).

E-cadherin endocytosis can occur in a clathrin-dependent (Palacios, et al. (2001) EMBO J. 20:4973-4986; Ivanov, et al. (2004) supra) or independent manner (Paterson, et al. (2003) J. Biol. Chem. 278:21050-21057). Calcium removal stimulates E-cadherin endocytosis by the clathrin-AP2 pathway (Ivano, et al. (2004) supra). As there is no known Tyr-Xaa-Xaa-φ (SEQ ID NO:4) sorting motif in the E-cadherin cytoplasmic domain, the interaction between PIPKIγ661 and E-cadherin may recruit AP2 for clathrin-dependent E-cadherin endocytosis. Additionally, Arf6 promotes E-cadherin internalization (Palacios, et al. (2002) Nat. Cell Biol. 4:929-936) and has been shown to associate with and stimulate the activity of PIPKIγ (Aikawa and Martin (2003) J. Cell Biol. 162:647-659; Aikawa and Martin (2005) Methods Enzymol. 404:422-431). Arf6, in cooperation with PI4,5P₂, was also shown to directly interact with and promote the recruitment of AP2 to the plasma membrane (Paleotti, et al. (2005) J. Biol. Chem. 280:21661-21666; Krauss, et al. (2003) J. Cell. Biol. 162:113-124). These combined results indicate that PIPKIγ661, E-cadherin, AP2 and Arf6 could cooperate to regulate E-cadherin internalization in epithelial cells.

PIPKIγ and p120 catenin associate specifically with dimeric E-cadherin cytoplasmic domain. Dimerization is an essential property of E-cadherin assembly driving adherens junction formation (Yap, et al. (1997) supra). Not wishing to be bound by theory, it is believed that the association of both PIPKIγ and p120 catenin with the E-cadherin dimer may be a mechanism to functionally regulate the association and could promote adherens junction formation by stimulating E-cadherin clustering. Since PIPKIγ specifically binds to E-cadherin dimers, the in situ PI4,5P2 generation resulting from this interaction may drive key local interactions such as actin reorganization (Janmey and Lindberg (2004) Nat. Rev. Mol. Cell Biol. 5:658-666). In turn actin assembly is important not only in adherens junction assembly but also for E-cadherin internalization/exocytosis (D'Souza-Schorey (2005) Trends Cell Biol. 15:19-26). The association of PIPKIγ with E-cadherin may well be crucial for downstream signaling as the small G-protein Rac and PI 3-kinase are activated by E-cadherin and both regulate the stability of adherens junctions by modulating actin assembly (Noren, et al. (2001) J. Biol. Chem. 276:33305-33308; Yap and Kovacs (2003) J. Cell Biol. 160:11-16; D'Souza-Schorey (2005) supra). PI 3-kinase requires PI4,5P₂ the product of PIP kinases for signaling, and Rho family small G-proteins regulate some PIPKI isoforms (Fruman, et al. (1998) Annu. Rev. Biochem. 67:481-507). As a result, PIPKIγ may also regulate adherens junction assembly through local cooperation with PI 3-kinase and small G-protein signaling.

The direct interaction of PIPKIγ with the E-cadherin dimer represents a novel association of a second messenger-generating enzyme with the classical cadherins. The generation of phosphoinositide messengers upon assembly of E-cadherin based adherens junctions has implications beyond simple control of E-cadherin trafficking. As E-cadherin is a major suppressor of invasion of epithelial tumors, the cell biological data indicates that PIPKIγ plays a similar role. In this regard, a loss of E-cadherin in human cancers was also found to be highly correlative with a loss of PIPKIγ.

When a gastric tissue sample from a Val832Met mutation cancer was immunostained, E-cadherin showed strong membrane staining in normal epithelia but diffused cytoplasmic staining in the adenocarcinoma cells. Unexpectedly, PIPKIγ also showed membrane staining in normal breast tissue but diffuse to weak staining in breast carcinoma cells (Table 3). When the Val832Met E-cadherin mutant was introduced into HEK 293 cells and its association with PIPKIγ assessed, it was found that the Val832Met E-cadherin mutant had substantially lower ability to bind PIPKIγ. However, as has been demonstrated in the art (Suriano, et al. (2003) supra), β-catenin binding to this E-cadherin mutant was normal. These combined results indicate that correct E-cadherin targeting is dependent upon its binding to PIPKIγ.

TABLE 3

| E-cadherin | PIPKIγ Staining Pattern | | | | | |
|---|---|---|---|---|---|---|
| staining | | | Weak | Strong | Basal | |
| pattern | Negative | Cytoplasmic | Mem. | Mem. | Layer | Total |
| Negative | 36 | 1 | 12 | 3 | 0 | 52 |
| Cytoplasmic | 7 | 0 | 4 | 4 | 0 | 15 |
| Nuclear | 8 | 1 | 2 | 1 | 0 | 12 |
| Nuclear + | 6 | 0 | 9 | 0 | 1 | 16 |
| Mem. | | | | | | |
| Weak Mem. | 42 | 3 | 50 | 22 | 5 | 122 |
| Strong Mem. | 36 | 17 | 64 | 34 | 1 | 152 |
| Total | 135 | 22 | 141 | 64 | 7 | 369 |

As the central component of adherens junctions, E-cadherin serves as a suppressor of invasion, and its functional elimination foretells a key step in invasion of many tumors. The loss of E-cadherin expression or function in carcinomas is a primary mechanism for disruption of adherens junctions and the induction of the epithelial-mesenchymal transition, which in turn leads to migration of cells from the primary tumor. The expression level of E-cadherin is often inversely correlated with tumor grade and stage. Furthermore, inactivating mutations in E-cadherin have been found in about 50% of breast lobular carcinomas (Kang and Massague (2004) Cell 118(3):277-9).

As the data presented herein indicate that PIPKIγ is required for normal E-cadherin function, the correlation of E-cadherin and PIPKIγ in normal breast and breast carcinomas was examined using tissue arrays. In normal breast tissue, PIPKIγ was observed at the plasma membrane with E-cadherin and PIPKIγ also showing a strong basal cell layer staining. In over 69% of breast carcinomas where E-cadherin was negative, PIPKIγ was also negative (Table 3). In a small fraction of breast carcinomas where E-cadherin membrane staining was totally lost but was present in or around the nucleus, PIPKIγ showed negative membrane staining. When the tissue array data were analyzed, a highly significant correlation between membranous E-cadherin (either strong or weak) and PIPKIγ staining (either strong or weak) was observed (P=0.00007). The rare nuclear (n=8) E-cadherin staining pattern correlated with negative PIPKIγ staining (P=0.008). A stronger correlation was observed when comparing all cases with negative or inappropriately localized E-cadherin with negative PIPKIγ staining (P=0.000001). In this regard, PIPKIγ staining was comparable with other breast cancer biomarkers (Table 4).

TABLE 4

| | Correlations/Spearman's | PIPKIγ | PIPKIγ |
|---|---|---|---|
| Biomarker | rho | Positive | Negative |
| PIPKIγ | Correlation Coefficient | 1 | −0.286 |
| Positive | Sig. (2-tailed) | | 0.000001 |
| | N | 438 | 438 |
| PIPKIγ | Correlation Coefficient | −0.286** | 1 |
| Negative | Sig. (2-tailed) | 0.000001 | |
| | N | 438 | 438 |
| HER1 | Correlation Coefficient | 0.242 | −0.131 |
| | Sig. (2-tailed) | 0.000005 | 0.015 |
| | N | 346 | 346 |
| HER2 | Correlation Coefficient | 0.164 | −0.102 |
| | Sig. (2-tailed) | 0.0026 | 0.062 |
| | N | 337 | 337 |
| HER1 or | Correlation Coefficient | 0.291 | −0.185 |
| HER2 | Sig. (2-tailed) | 0.000001 | 0.001 |
| | N | 299 | 299 |
| HER3 | Correlation Coefficient | −0.041 | −0.055 |
| | Sig. (2-tailed) | 0.496 | 0.365 |
| | N | 278 | 278 |
| HER3_01 | Correlation Coefficient | −0.013 | −0.069 |
| | Sig. (2-tailed) | 0.835 | 0.253 |
| | N | 278 | 278 |
| ER | Correlation Coefficient | −0.327 | 0.179 |
| | Sig. (2-tailed) | 0.000001 | 0.0015 |
| | N | 324 | 324 |
| p53 | Correlation Coefficient | 0.269 | −0.135 |
| | Sig. (2-tailed) | 0.000002 | 0.0178 |
| | N | 309 | 309 |

**Correlation is significant at the 0.01 level (2-tailed).

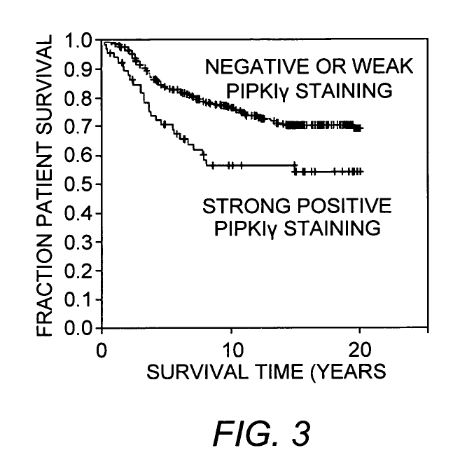

Moreover, PIPKIγ staining was indicative of patient survival (FIG. 3). These combined results indicate a very high correlation between the correct E-cadherin targeting and loss of E-cadherin, and the loss or mis-targeting of PIPKIγ. These data reveal a key role of PIPKIγ in the assembly of E-cadherin contacts. Since E-cadherin is a suppressor of invasion, PIPKIγ appears to have a similar role.

Having demonstrated that PIPKIγ directly binds cadherins and μ-subunits of AP complexes and functions to regulate cadherin clustering and trafficking (i.e., exocytosis and endocytosis), the present invention is a method for identifying agents which modulate these activities of PIPKIγ. The method involves contacting a PIPKIγ with a test agent in the presence of a cadherin or a μ-subunit and determining whether the agent modulates the trafficking of the PIPKIγ or the cadherin, or binding of the PIPKIγ with the cadherin or the μ-subunit as compared to a control, wherein a change in trafficking or binding as compared to a control indicates that the agent modulates the trafficking or binding activity of a PIPKIγ. As used herein, an agent which modulates the trafficking or binding activity of a PIPKIγ includes an agent which stimulates, enhances or activates PIPKIγ activity as well as an agent which inhibits, reduces or decreases the activity of PIPKIγ.

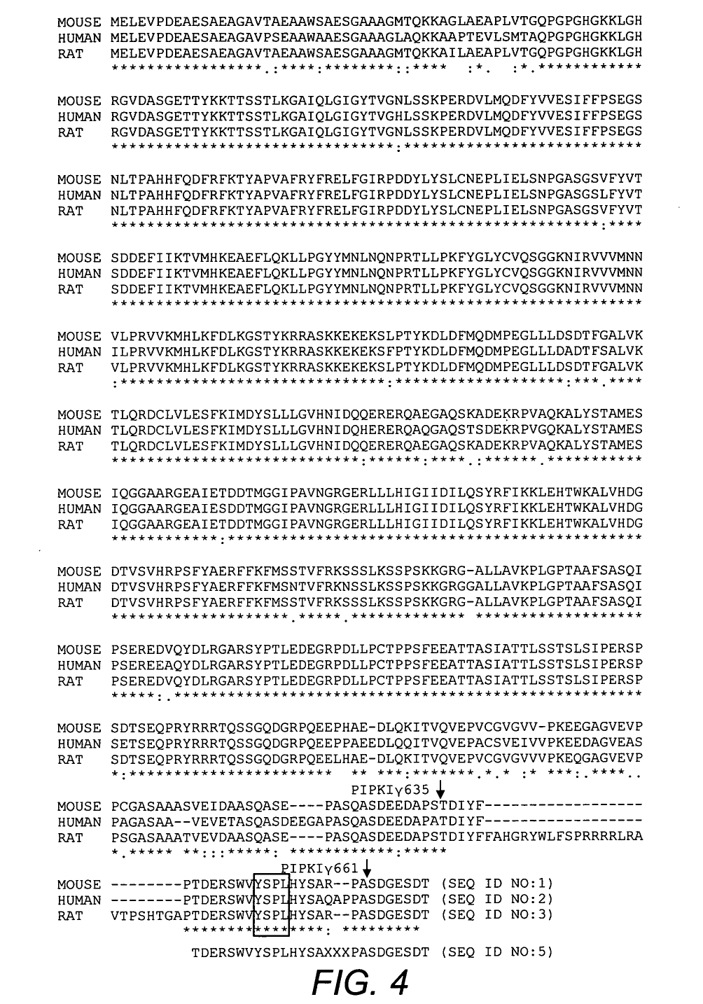

In the context of the assays disclosed herein, a PIPKIγ is intended to include a full-length PIPKIγ (e.g., 668 amino acid human protein; see FIG. 4) or a splice variants or fragments thereof which bind to a cadherin or a μ-subunit and functions to regulate cadherin trafficking. In particular embodiments of the invention, a splice variant of PIPKIγ encompasses PIPKIy661 (e.g., the 661 N-terminal amino acid residues of human PIPKIγ protein of SEQ ID NO:2; FIG. 4) or PIPKIγ635 (e.g., the 635 N-terminal amino acid residues of human PIPKIγ protein of SEQ ID NO:2; FIG. 4). In other embodiments, a fragment of a PIPKIγ includes, but is not limited to, the C-terminal 25 amino acid residues of PIPKIγ661, i.e., Thr-Asp-Glu-Arg-Ser-Trp-Val-Tyr-Ser-Pro-Leu-His-Tyr-Ser-Ala-Xaa,-Xaa₂-Xaa₃-Pro-Ala-Ser-Asp-Gly-Glu-Ser-Asp-Thr (SEQ ID NO:5, wherein Xaa₁ is Arg or Gln and Xaa₂ and Xaa₃ are absent or any amino acid residue); Thr-Asp-Glu-Arg-Ser-Trp-Val-Tyr-Ser-Pro-Leu-His-Tyr-Ser-Ala-Arg-Pro-Ala-Ser-Asp-Gly-Glu-Ser-Asp-Thr (SEQ ID NO:12); or a μ-subunit interacting peptide thereof (e.g., Cys-Asp-Glu-Arg-Ser-Trp-Val-Tyr-Ser-Pro-Leu-His-Tyr-Ser-Ala-Arg; SEQ ID NO:13). In some embodiments, a fragment of a PIPKIγ contains the tetrapeptide sequence Tyr-Xaa-Xaa-φ (SEQ ID NO:4), wherein “Xaa” is any amino acid residue and φ represents a hydrophobic residue, e.g., Leu, Ile, Val, Met, Phe, Trp, Tyr, Thr, or Ala (Bonifacino & Traub (2003) Annu. Rev. Biochem. 72:395-447). Other embodiments embrace a fragment of a PIPKIγ containing the tetrapeptide sequence Tyr-Ser-Pro-Leu (SEQ ID NO:11).

For use in accordance with the assays of the present invention, a cadherin is intended to include a full-length E-cadherin, N-cadherin, P-cadherin or VE-cadherin (see Table 5), or any fragment which binds to PIPKIγ, whether as a monomer or dimer.

TABLE 5

| | | GENBANK |
|---|---|---|
| Cadherin | Source | Accession No. |
| E-cadherin | Homo sapiens | CAA78353 |
| | Sus scrofa | AAB87474.1 |
| | Mus musculus | NP_033994.1 |
| | Rattus norvegicus | BAA84920.1 |
| N-cadherin | Homo sapiens | AAA03236 |
| | Bos taurus | CAA37677.1 |
| | Mus musculus | BAA23549.1 |
| | Rattus norvegicus | AAF87057.1 |
| P-cadherin | Homo sapiens | CAA45177.1 |
| | Bos taurus | CAA37676.1 |
| | Mus musculus | AAH98459.1 |
| VE-cadherin | Homo sapiens | CAA56306 |
| | Sus scrofa | BAB82983.1 |
| | Mus musculus | CAA58782.2 |

In particular embodiments of the invention, a fragment of a cadherin encompasses amino acid residues 837 to 847 of E-cadherin (i.e., the conserved sequence Gly-Ser-Gly-Ser-Glu-Ala-Ala-Ser-Leu-Ser-Ser; SEQ ID NO:7), N-cadherin (i.e., the conserved sequence Gly-Ser-Gly-Ser-Thr-Ala-Gly-Ser-Leu-Ser-Ser; SEQ ID NO:8), P-cadherin (i.e., the conserved sequence Gly-Ser-Gly-Ser-Asp-Ala-Ala-Ser-Leu-Ser-Ser; SEQ ID NO:9) or VE-cadherin (i.e., Gly-Ser-Glu-Ser-Ile-Ala-Glu-Ser-Leu-Ser-Ser; SEQ ID NO:10).

A μ-subunit protein which can be used within the scope of the invention includes a full-length μ1B protein (see e.g., Nakatsu, et al. (1999) Cytogenet. Cell Genet. 87:53-58) or μ2 protein (see e.g., Thurieau, et al. (1988) DNA 7(10):663-9) as provided in Table 6, or a fragment thereof which binds to PIPKIγ.

TABLE 6

| | | GENBANK |
|---|---|---|
| μ-subunit | Source | Accession No. |
| μ1B protein | Homo sapiens | NP_005489 |
| | Mus musculus | NP_033808 |
| μ2 protein | Homo sapiens | NP_004059 |
| | Rattus norvegicus | NP_446289 |

The μ-subunit protein can be used alone or as a component of an adaptor protein complex (e.g., AP1 or AP2). In some embodiments, a fragment of a μ-subunit protein encompasses the C-terminal 270 amino acid residues of a μ1B-subunit or μ2-subunit protein.

The assay of the present invention can advantageously be carried out in vitro or in vivo. Generally, either an in vitro or in vivo assay is used to determine the binding activity of a PIPKIγ (i.e., binding between a PIPKIγ and a cadherin or a PIPKIγ and a μ-subunit) in the presence of the test agent and an in vivo assay is used to determine the trafficking activity of PIPKIγ in the presence of a test agent.

For in vitro binding assays, a PIPKIγ, cadherin or μ-subunit protein or fragment thereof can be recombinantly-produced or chemically-synthesized so long as the protein or fragment thereof functions in a manner similar to the reference molecule to achieve a desired result. Thus, a functional PIPKIγ, cadherin or μ-subunit encompasses derivatives, homologues, orthologs and analogues of those proteins including any single or multiple amino acid additions, substitutions, and/or deletions occurring internally or at the amino or carboxy termini thereof so long as binding activity remains.

Methods for producing recombinant proteins such as PIPKIγ, cadherin or μ-subunit proteins are well-known in the art. In general, nucleic acid sequences encoding PIPKIγ, cadherin or μ-subunit are incorporated into a recombinant expression vector in a form suitable for expression of the proteins in a host cell (e.g., a prokaryotic, yeast, insect, mammalian, or plant cell). A suitable form for expression provides that the recombinant expression vector includes one or more regulatory sequences operatively-linked to the nucleic acids encoding PIPKIγ, cadherin or μ-subunit in a manner which allows for transcription of the nucleic acids into mRNA and translation of the mRNA into the protein. Regulatory sequences can include promoters, enhancers and other expression control elements (e.g., polyadenylation signals). Such regulatory sequences are known to those skilled in the art and are described in Goeddel D. D., ed., Gene Expression Technology, Academic Press, San Diego, Calif. (1991). It should be understood that the design of the expression vector can depend on such factors as the choice of the host cell to be transfected and/or the level of expression required.

A PIPKIγ, cadherin or μ-subunit protein can be expressed not only directly, but also as a fusion protein with a heterologous polypeptide, i.e. a signal sequence for secretion and/or other polypeptide which will aid in the purification of a PIPKIγ, cadherin or μ-subunit. In certain applications, the heterologous polypeptide has a specific cleavage site to remove the heterologous polypeptide from the PIPKIγ, cadherin or μ-subunit protein.

A signal sequence can also be a component of the vector and should be one that is recognized and processed (i.e., cleaved by a signal peptidase) by the host cell. For production in a prokaryote, a prokaryotic signal sequence from, for example, alkaline phosphatase, penicillinase or heat-stable enterotoxin II leaders can be used. For yeast secretion, one can use, e.g., the yeast invertase, alpha factor, acid phosphatase leaders, the Candida albicans glucoamylase leader (EP 362,179), or the like (see, for example WO 90/13646). In mammalian cell expression, signal sequences from secreted polypeptides of the same or related species, as well as viral secretory leaders, for example, the herpes simplex glycoprotein D signal may be used.

Other useful heterologous polypeptides which can be fused to a PIPKIγ, cadherin or μ-subunit protein include those which increase expression or solubility of the fusion protein or aid in the purification of the fusion protein by acting as a ligand in affinity purification. Typical fusion expression vectors include those exemplified herein (i.e., fusion with c-Myc, His, or GST) as well as PMAL and pTYB vectors (New England Biolabs, Beverly, Mass.) and pRIT5 (Pharmacia Biotech, Piscataway, N.J.) which fuse maltose E binding protein, intein/chitin binding domain or protein A, respectively, to the target recombinant protein.

A PIPKIγ, cadherin or μ-subunit protein is expressed in a cell by introducing nucleic acid sequences encoding a PIPKIγ, cadherin or μ-subunit protein into a host cell, wherein the nucleic acids are in a form suitable for expression of a PIPKIγ, cadherin or μ-subunit protein in the host cell. Alternatively, nucleic acid sequences encoding a PIPKIγ, cadherin or μ-subunit protein which are operatively-linked to regulatory sequences (e.g., promoter sequences) but without additional vector sequences can be introduced into a host cell. As used herein, a host cell is intended to include any prokaryotic or eukaryotic cell or cell line so long as the cell or cell line is not incompatible with the protein to be expressed, the selection system chosen or the fermentation system employed.

Eukaryotic cells or cell lines which can be used to produce a PIPKIγ, cadherin or μ-subunit protein include mammalian cell lines as well as non-mammalian cells. Exemplary mammalian cell lines include, but are not limited to, those exemplified herein as well as CHO dhfr- cells (Urlaub and Chasin (1980) Proc. Natl. Acad. Sci. USA 77:4216-4220), 293 cells (Graham, et al. (1977) J. Gen. Virol. 36:59), or myeloma cells like SP2 or NSO (Galfre and Milstein (1981) Meth. Enzymol. 73(B):3-46). A variety of non-mammalian eukaryotic cells can be used as well, including insect (e.g,. Spodoptera frugiperda), yeast (e.g., S. cerevisiae, Schizosaccharomyces pombe, Pichia pastoris, Kluveromyces lactis, Hansenula polymorpha, and Candida albicans), fungal cells (e.g., Neurospora crassa, Aspergillus nidulins, Aspergillus fumigatus) and plant cells.

While any prokaryotic cell can be used to recombinantly produce a PIPKIγ, cadherin or μ-subunit protein, Escherichia coli is the most common prokaryotic expression system. Strains which can be used to maintain expression plasmids include, but are not limited to, JM103, JM105, and JM107. Exemplary E. coli strains for protein production include W3110 (ATCC 27325), E. coli B, E. coli X1776 (ATCC 31537), E. coli BL21 (Amersham Biosciences, Piscataway, N.J.), E. coli ER5266 (New England Biolabs, Beverly, Mass.) and E. coli 294 (ATCC 31446).

For production of a PIPKIγ, cadherin or μ-subunit protein in recombinant prokaryotic expression vectors it is contemplated that protein expression is regulated by promoters such as the beta-lactamase (penicillinase) and lactose promoter systems (Chang, et al. (1978) Nature 275:615; Goeddel, et al. (1979) Nature 281:544), a tryptophan (trp) promoter system (Goeddel, et al. (1980) Nucl. Acids Res. 8:4057; EP 36,776) the tac promoter (De Boer, et al. (1983) Proc. Natl. Acad. Sci. USA 80:21) or pL of bacteriophage 1. These promoters and Shine-Dalgarno sequence can be used for efficient expression of a PIPKIγ, cadherin or μ-subunit protein in prokaryotes.

Eukaryotic microbes such as yeast can also be transformed with suitable vectors containing nucleic acids encoding a PIPKIγ, cadherin or μ-subunit protein. Saccharomyces cerevisiae is the most commonly studied lower eukaryotic host microorganism, although a number of other species already mentioned are commonly available. Yeast vectors generally contain an origin of replication from the 2 micron yeast plasmid or an autonomously replicating sequence (ARS), a promoter, nucleic acid sequences encoding a PIPKIγ, cadherin or μ-subunit protein, sequences for polyadenylation and transcription termination, and nucleic acid sequences encoding a selectable marker. Exemplary plasmids include YRp7 (Stinchcomb, et al. (1979) Nature 282:39; Kingsman, et al. (1979) Gene 7:141; Tschemper, et al. (1980) Gene 10:157), pYepSecl (Baldari, et al. (1987) EMBO J. 6:229-234), pMFa (Kurjan and Herskowitz (1982) Cell 30:933-943), pJRY88 (Schultz, et al. (1987) Gene 54:113-123), and pYES2 (INVITROGENT™ Corporation, San Diego, Calif.). These plasmids contain genes such as trpl, which provides a selectable marker for a mutant strain of yeast lacking the ability to grow in the presence of tryptophan, for example ATCC No. 44076 or PEP4-1 (Jones (1977) Genetics 85:12). The presence of the trpl lesion in the yeast host cell genome then provides an effective environment for detecting transformation by growth in the absence of tryptophan.

Suitable sequences for promoting PIPKIγ, cadherin or μ-subunit protein expression in yeast vectors include the promoters for metallothionein, 3-phosphoglycerate kinase (Hitzeman, et al. (1980) J. Biol. Chem. 255:2073) or other glycolytic enzymes (Hess, et al. (1968) J. Adv. Enzyme Reg. 7:149; Holland, et al. (1978) Biochemistry 17:4900), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are further disclosed in EP 73,657.

When the host cell is from an insect (e.g., Spodoptera frugiperda cells), expression vectors such as the baculovirus expression vector (e.g., vectors derived from Autographa californica MNPV, Trichoplusia ni MNPV, Rachiplusia ou MNPV, or Galleria ou MNPV, as described in U.S. Pat. Nos. 4,745,051 and 4,879,236) can be employed to express a PIPKIγ, cadherin or μ-subunit protein. In general, a baculovirus expression vector encompasses a baculovirus genome containing nucleic acid sequences encoding a PIPKIγ, cadherin or μ-subunit protein inserted into the polyhedrin gene at a position ranging from the polyhedrin transcriptional start signal to the ATG start site and under the transcriptional control of a baculovirus polyhedrin promoter.

In plant cells, expression systems are often derived from recombinant Ti and Ri plasmid vector systems. In the co-integrate class of shuttle vectors, the gene of interest is inserted by genetic recombination into a non-oncogenic Ti plasmid that contains both the cis-acting and trans-acting elements required for plant transformation. Exemplary vectors include the pMLJ1 shuttle vector (DeBlock, et al. (1984) EMBO J. 3:1681-1689) and the non-oncogenic Ti plasmid pGV2850 (Zambryski, et al. (1983) EMBO J. 2:2143-2150). In the binary system, the gene of interest is inserted into a shuttle vector containing the cis-acting elements required for plant transformation. The other necessary functions are provided in trans by the non-oncogenic Ti plasmid. Exemplary vectors include the pBIN19 shuttle vector (Bevan (1984) Nucl. Acids Res. 12:8711-8721) and the non-oncogenic Ti plasmid pAL4404 (Hoekema, et al. (1983) Nature 303:179-180) and derivatives thereof.

Promoters used in plant expression systems are typically derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV).

In mammalian cells the recombinant expression vector can be a plasmid. Alternatively, a recombinant expression vector can be a virus, or a portion thereof, which allows for expression of a nucleic acid introduced into the viral nucleic acid. For example, replication-defective retroviruses, adenoviruses and adeno-associated viruses can be used. Protocols for producing recombinant retroviruses and for infecting cells in vitro or in vivo with such viruses can be found in Current Protocols in Molecular Biology, Ausubel, F. M. et al. (eds.) John Wiley & Sons, (1996), Section 9 and other standard laboratory manuals. Examples of suitable retroviruses include, but are not limited to, pLJ, pZIP, pWE and pEM, which are well-known to those skilled in the art. Examples of suitable packaging virus lines include, but are not limited to, ψCrip, ψCre, ψ2 and ψAm. The genome of adenovirus can be manipulated such that it encodes and expresses PIPKIγ, cadherin or μ-subunit protein but is inactivated in terms of its ability to replicate in a normal lytic viral life cycle (Berkner, et al. (1988) BioTechniques 6:616; Rosenfeld, et al. (1991) Science 252:431-434; Rosenfeld, et al. (1992) Cell 68:143-155). Suitable adenoviral vectors derived from the adenovirus strain Ad type 5 or other strains of adenovirus (e.g., Ad2, Ad3, Ad7 etc.) are well-known to those skilled in the art. Alternatively, an adeno-associated virus vector such as that taught by Tratschin, et al. ((1985) Mol. Cell. Biol. 5:3251-3260) can be used to express a PIPKIγ, cadherin or μ-subunit protein.

In mammalian expression systems, the regulatory sequences are often provided by the viral genome. Commonly used promoters are derived from polyoma, Adenovirus 2, cytomegalovirus and Simian Virus 40. For example, the human cytomegalovirus IE promoter (Boshart, et al. (1985) Cell 41:521-530), HSV-Tk promoter (McKnight, et al. (1984) Cell 37:253-262) and P-actin promoter (Ng, et al. (1985) Mol. Cell. Biol. 5:2720-2732) can be useful in the expression of a PIPKIγ, cadherin or μ-subunit protein in mammalian cells. Alternatively, the regulatory sequences of the recombinant expression vector can direct expression of a PIPKIγ, cadherin or μ-subunit protein preferentially in a particular cell-type, i.e., tissue-specific regulatory elements can be used. Examples of tissue-specific promoters which can be used include, but are not limited to, the albumin promoter (liver-specific; Pinkert, et al. (1987) Genes Dev. 1:268-277), lymphoid-specific promoters (Calame and Eaton (1988) Adv. Immunol. 43:235-275), promoters of T cell receptors (Winoto and Baltimore (1989) EMBO J. 8:729-733) and immunoglobulins (Banerji, et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci USA 86:5473-5477), pancreas-specific promoters (Edlund, et al. (1985) Science 230:912-916), and mammary gland-specific promoters (e.g., milk whey promoter; U.S. Pat. No. 4,873,316; EP 264,166). Developmentally-regulated promoters are also encompassed, for example the murine hox promoters (Kessel and Gruss (1990) Science 249:374-379) and the α-fetoprotein promoter (Camper and Tilghman (1989) Genes Dev. 3:537-546).

Nucleic acid sequences encoding a PIPKIγ, cadherin or μ-subunit protein can be introduced into cells growing in culture in vitro by conventional transformation techniques (e.g., calcium phosphate precipitation, DEAE-dextran transfection, electroporation, etc.). Nucleic acids can also be transferred into cells in vivo, for example by application of a delivery mechanism suitable for introduction of nucleic acid into cells in vivo, such as retroviral vectors (see e.g., Ferry, et al. (1991) Proc. Natl. Acad. Sci. USA 88:8377-8381; Kay, et al. (1992) Hum. Gene Ther. 3:641-647), adenoviral vectors (see e.g., Rosenfeld (1992) Cell 68:143-155; Herz and Gerard (1993) Proc. Natl. Acad. Sci. USA 90:2812-2816), receptor-mediated DNA uptake (see e.g., Wu and Wu (1988) J. Biol. Chem. 263:14621; Wilson, et al. (1992) J. Biol. Chem. 267:963-967; U.S. Pat. No. 5,166,320), direct injection of DNA uptake (see e.g., Acsadi, et al. (1991) Nature 334:815-818; Wolff, et al. (1990) Science 247:1465-1468), Agrobacterium-mediated transformation, cell fusion, or ballistic bombardment (see e.g., Cheng, et al. (1993) Proc. Natl. Acad. Sci. USA 90:4455-4459; Zelenin, et al. (1993) FEBS Let. 315:29-32). Suitable methods for transforming host cells can also be found in Sambrook, et al. (Molecular Cloning: A Laboratory Manual, 3rd Edition, Cold Spring Harbor Laboratory Press (2000)) and other laboratory manuals.

The number of host cells transformed with a nucleic acid sequence encoding a PIPKIγ, cadherin or μ-subunit protein will depend, at least in part, upon the type of recombinant expression vector used and the type of transformation technique used. Nucleic acids can be introduced into a host cell transiently, or more typically, for long-term expression of a PIPKIγ, cadherin or μ-subunit protein, the nucleic acid sequence is stably integrated into the genome of the host cell or remains as a stable episome in the host cell. Plasmid vectors introduced into mammalian cells are typically integrated into host cell DNA at only a low frequency. In order to identify these integrants, a gene that contains a selectable marker (e.g., drug resistance) is generally introduced into the host cells along with the nucleic acids of interest. Suitable selectable markers include those which confer resistance to certain drugs, such as G418 and hygromycin. Selectable markers can be introduced on a separate plasmid from the nucleic acids of interest or introduced on the same plasmid. Host cells transfected with nucleic acid sequences encoding a PIPKIγ, cadherin or μ-subunit protein (e.g., a recombinant expression vector) and a gene for a selectable marker can be identified by selecting for cells expressing the selectable marker. For example, if the selectable marker encodes a gene conferring neomycin resistance, host cells which have taken up the nucleic acid sequences of interest can be selected for G418 resistance. Cells that have incorporated the selectable marker gene will survive, while the other cells die.

Nucleic acid sequences encoding a PIPKIγ, cadherin or μ-subunit protein can also be transferred into a fertilized oocyte of a non-human animal to create a transgenic animal which expresses a PIPKIγ, cadherin or μ-subunit protein in one or more cell-types. A transgenic animal is an animal having cells that contain a transgene, wherein the transgene was introduced into the animal or an ancestor of the animal at a prenatal, e.g., an embryonic, stage. A transgene is a DNA which is integrated into the genome of a cell from which a transgenic animal develops and which remains in the genome of the mature animal, thereby directing the expression of an encoded gene product in one or more cell-types or tissues of the transgenic animal. Exemplary non-human animals include, but are not limited to, mice, goats, sheep, pigs, cows or other domestic farm animals. Such transgenic animals are useful, for example, for large-scale production of a PIPKIγ, cadherin or μ-subunit protein (gene pharming) or for basic research investigations.

A transgenic animal can be created, for example, by introducing a nucleic acid sequence encoding a PIPKIγ, cadherin or μ-subunit protein, typically linked to appropriate regulatory sequences, such as a constitutive or tissue-specific enhancer, into the male pronuclei of a fertilized oocyte, e.g., by microinjection, and allowing the oocyte to develop in a pseudopregnant female foster animal. Intron sequences and polyadenylation signals can also be included in the transgene to increase the efficiency of expression of the transgene. Methods for generating transgenic animals, particularly animals such as mice, have become conventional in the art and are described, for example, in U.S. Pat. Nos. 4,736,866 and 4,870,009. A transgenic founder animal can be used to breed additional animals carrying the transgene.

Once a cell line or transgenic animal is established, a PIPKIγ, cadherin or μ-subunit protein can be recovered from culture medium or milk as a secreted polypeptide, or recovered from host cell lysates when directly expressed without a secretory signal. When a PIPKIγ, cadherin or μ-subunit protein is expressed in a recombinant cell other than one of human origin, the PIPKIγ, cadherin or μ-subunit protein is generally free of proteins or polypeptides of human origin. However, it may be necessary to purify the PIPKIγ, cadherin or μ-subunit protein from recombinant cell proteins or polypeptides to obtain preparations that are substantially homogeneous as to PIPKIγ, cadherin or μ-subunit protein. As a first step, the culture medium or lysate is centrifuged to remove particulate cell debris. The membrane and soluble protein fractions are then separated. The PIPKIγ, cadherin or μ-subunit protein can then be purified from the soluble protein fraction. Depending on whether the PIPKIγ, cadherin or μ-subunit protein is fused to a heterologous polypeptide or not, the PIPKIγ, cadherin or μ-subunit protein can be purified from contaminant soluble proteins and polypeptides using various methods including, but not limited to, fractionation on immunoaffinity or ion-exchange columns; ethanol precipitation; chitin column chromatography, reverse phase HPLC; chromatography on silica or on a cation-exchange resin such as DEAE; chromatofocusing; SDS-PAGE; ammonium sulfate precipitation; gel filtration using, for example, SEPHADEX® G-75; ligand affinity chromatography, and protein A SEPHAROSE® columns to remove contaminants such as IgG.

In addition to recombinant production, a PIPKIγ, cadherin or μ-subunit protein, or fragments thereof can be produced by direct peptide synthesis using solid-phase techniques (Merrifield J. (1963) J. Am. Chem. Soc. 85:2149-2154). Protein synthesis can be performed using manual techniques or by automation. Automated synthesis can be achieved, for example, using Applied Biosystems 431A Peptide Synthesizer (Perkin Elmer, Boston, Mass.). Various fragments of a PIPKIγ, cadherin or μ-subunit protein can be chemically-synthesized separately and combined using chemical methods to produce the full-length molecule.

Whether recombinantly-produced or chemically-synthesized, a PIPKIγ, cadherin or μ-subunit protein or fragments thereof can be further modified for use in the screening assays of the invention. For example, the proteins or fragments thereof can be glycosylated, phosphorylated (e.g., tyrosine 644 or 649 of PIPKIγ661) or fluorescently-tagged using well-known methods.

In an in vitro screening assay to identify an agent that modulates the binding activity of a PIPKIγ, the step of determining whether the agent modulates the binding of the PIPKIγ with the cadherin or the μ-subunit as compared to a control is carried out by detecting and measuring (e.g., the affinity) the binding of a PIPKIγ to a cadherin or a μ-subunit. The detection and measurement of this binding interaction will be dependent on the type of screening assay performed and the labels used. One method for detecting and measuring binding between two proteins is exemplified herein (i.e., GST pull-down or immunoprecipitation) and other such methods are well-known in the art. As an example, a μ-subunit-interacting peptide of PIPKIγ661, i.e., Cys-Asp-Glu-Arg-Ser-Trp-Val-Tyr-Ser-Pro-Leu-His-Tyr-Ser-Ala-Arg (SEQ ID NO:13), having the cysteine residue modified, e.g., by conventional thiol or thiolester linkages, with a label (e.g., a fluorescent label) can be used in a μ-subunit binding assay wherein detection of the binding interaction is carried out by measuring changes in rotational motion using fluorescent emission anisotropy. In this assay, an agent which is an inhibitor of the interaction between PIPKIγ661 and μ-subunit blocks binding of a peptide of SEQ ID NO:13 and changes the fluorescent emission anisotropy as compared to a control (e.g., binding in the absence of the inhibitor).

Exemplary fluorescent probes which can be attached to the N-terminus of a PIPKIγ661 peptide are well-known in the art and include, but are not limited to, α-Phycoerythrin, Green Fluorescent Protein, Phycocyanine, Allophycocyanine, Tricolor, AMCA, AMCA-S, AMCA, BODIPY FL, BODIPY 493/503, BODIPY FL Br2, BODIPY R6G, BODIPY 530/550, BODIPY TMR, BODIPY 558/568, BODIPY 564/570, BODIPY 576/589, BODIPY 581/591, BODIPY TR, Cascade Blue, CI-NERF, Dansyl, Dialkylaminocoumarin, 4′,6′-Dichloro-2′,7′-dimethoxy-fluorescein, 2′,7′-dichloro-fluorescein, Cy3, Cy5, Cy7, DM-NERF, Eosin, Eosin F3S, Erythrosin, Fluorescein, Fluorescein Isothiocyanate Hydroxycoumarin, Isosulfan Blue, Lissamine Rhodamine B, Malachite Green, Methoxycoumarin, Napthofluorecein, NBD, Oregon Green 488, Oregon Green 500, Oregon Green 514, Propidium Iodide Phycoerythrin, PyMPO, Pyrene, Rhodamine 6G, Rhodamine Green, Rhodamine Red, Rhodol Green, 2′,4′,5′,7′-Tetrabromosulfonefluorescein, Tetramethyl-rhodamine, Texas Red, X-rhodamine; Lucifer Yellow and the like. Detection of changes in fluorescence can be carried out using such methods as fluorescence spectroscopy, fluorescence resonance energy transfer (FRET), fluorescent lifetime imaging (FLIM) (Lakowicz, et al. (1992) Anal. Biochem. 202:316-330), or fluorescence polarization.

Alternatively, an in vivo assay can be used to determine whether a test agent modulates the binding activity of a PIPKIγ with a cadherin or a μ-subunit. By way of illustration, a two-hybrid assay is contemplated where the test agent is contacted with a cell expressing a PIPKIγ and a cadherin or a μ-subunit, wherein the PIPKIγ is fused to, e.g., a DNA-binding domain and the cadherin or μ-subunit is fused to an activation domain. When the cadherin or μ-subunit is bound to the PIPKIγ, reporter protein expression is induced. If the test agent disrupts the binding of the cadherin or μ-subunit to the PIPKIγ, reporter protein expression is blocked.

A host cell transformed with nucleic acid sequences encoding a PIPKIγ, cadherin or μ-subunit protein can also be used in vivo screening assays for determining whether a test agent modulates the trafficking activity of PIPKIγ. As used herein, the trafficking activity of PIPKIγ relates to the finding that PIPKIγ binds both μ-subunit and cadherin and mediates the exocytosis and endocytosis of cadherin. As exemplified herein, the trafficking activity of PIPKIγ can be determined by immunofluorescent detection of the subcellular location of PIPKIγ or cadherin in the presence of a test agent and absence of a test agent (i.e., control). Alternatively, PIPKIγ or cadherin can be labeled with a fluorescent tag (e.g., tagged with GFP or a biotin peptide) and localization can be ascertained by fluorescent microscopy. Further, it is contemplated that cells contacted with a test agent can be fractionated according to well-established centrifugation methods and subcellular fractions (e.g., the cytosolic and membrane fractions) analyzed for the presence or absence of a PIPKIγ or a cadherin, e.g., using antibodies or fluorescent tags. Agents identified in accordance with this in vivo assay can include agents which directly interact with PIPKIγ, cadherin or μ-subunit proteins or modulate the expression of nucleic acids encoding PIPKIγ, cadherin or μ-subunit protein thereby changing the amount of the protein in the cell and therefore trafficking patterns.

The in vitro and in vivo screening assays disclosed herein can be performed in any format that allows rapid preparation and processing of multiple reactions such as in, for example, multi-well plates of the 96-well variety. Stock solutions of test agents as well as assay components are prepared manually and all subsequent pipetting, diluting, mixing, washing, incubating, sample readout and data collecting is done using commercially available robotic pipetting equipment, automated work stations, and analytical instruments for detecting the signal generated by the assay.

In addition to PIPKIγ, cadherin or μ-subunit protein, a variety of other reagents can be included in the screening assays. These include reagents like salts, neutral proteins, e.g., albumin, detergents, etc. which can be used to facilitate optimal protein-protein binding and/or reduce non-specific or background interactions. Also, reagents that otherwise improve the efficiency of the assay, such as protease inhibitors, nuclease inhibitors, anti-microbial agents, and the like can be used. The mixture of components can be added in any order that provides for the requisite binding.

When assaying test agents in the binding and trafficking assays of the instant invention, it is desirable that a control be included for comparison. For example, a control can be a test reaction which lacks the test agent or a control reaction can be test reaction which contains a known agent which has a high affinity for binding and inhibiting the interaction between PIPKIγ and cadherin or PIPKIγ and μ-subunit proteins.

Test agents which can be screened in accordance with the methods of the present invention are generally derived from libraries of agents or compounds. Such libraries can contain either collections of pure agents or collections of agent mixtures. Examples of pure agents include, but are not limited to, proteins, antibodies, aptamers, polypeptides, peptides, nucleic acids, oligonucleotides, siRNA, carbohydrates, lipids, synthetic or semi-synthetic chemicals, and purified natural products. Examples of agent mixtures include, but are not limited to, extracts of prokaryotic or eukaryotic cells and tissues, as well as fermentation broths and cell or tissue culture supernates. In the case of agent mixtures, the methods of this invention are not only used to identify those crude mixtures that possess the desired activity, but also provide the means to monitor purification of the active agent from the mixture for characterization and development as a therapeutic drug. In particular, the mixture so identified can be sequentially fractionated by methods commonly known to those skilled in the art which can include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained.

Additional screens, such as well-established computational screens, are also contemplated for use in conjunction with the screening method disclosed herein. Such screens could employ using the agents disclosed herein as lead compounds for the generation of libraries of compounds which modulate the activity of PIPKIγ.

Exemplary agents of the instant invention include siRNA molecules, including but not limited to those set forth in SEQ ID NO:14, 15, or 16, as well as peptides which could compete for binding of full-length PIPKIγ with a cadherin or a μ-subunit, e.g., peptides set forth herein as SEQ ID NO:4, 5, 7, 8, 9, 10, 11, 12, or 13.

Agents identified in accordance with the assay method of the present invention will be useful in various applications including blocking endocytosis of E-cadherin (e.g., blocking PIPKIγ/μ2 or PIPKIγ/E-cadherin interactions), thereby maintaining cell-cell contacts in epithelial cells and decreasing transition from the epithelial to the mesenchymal phenotype associated with various cancers derived from epithelial cells (e.g., breast, colon, ovarian and prostate cancers); cancer prevention and treatment; facilitating exocytosis and/or clustering of E-cadherin, e.g., in cells with reduced PIPKγ expression or activity; or decreasing expression (e.g., using the siRNA of SEQ ID NO:14, 15, or 16) or activity of PIPKγ to transiently enhance epithelial depolarization thereby facilitating wound healing, immune responses, or neuronal development. Having demonstrated that PIPKγ is a member of the AP1B/AP2 complex, PIPKγ will also likely be involved in the trafficking of other proteins to which it binds including N-cadherin, VE-cadherin, and P-cadherin, and thus agents which activate or inhibit PIPKγ activity or expression will be useful in modulating the trafficking of these binding proteins and therefore their function (e.g., in embryonic development, cell motility, and cancer progression).

To evaluate the efficacy of an agent identified using the screening method of the invention, one of skill will appreciate that a model system of any particular disease or disorder involving PIPKγ activity or cadherin localization can be utilized to evaluate the adsorption, distribution, metabolism and excretion of a compound as well as its potential toxicity in acute, sub-chronic and chronic studies.

The use of an agent identified in accordance with the assay method of the present invention in the prevention or treatment of a disease or condition involving PIPKγ activity or cadherin localization typically involves the steps of first identifying a patient at risk of having or having a disease or disorder involving PIPKγ activity or cadherin localization (e.g., cancer invasiveness or metastasis, wound healing, immune responses, or neuronal development). Once such an individual is identified using, for example, standard clinical practices, said individual is administered a pharmaceutical composition containing an effective amount of an agent identified in the screening methods of the invention. In most cases this will be a human being, but treatment of agricultural animals, e.g., livestock and poultry, and companion animals, e.g., dogs, cats and horses, is expressly covered herein. The selection of the dosage or effective amount of an agent is that which has the desired outcome of reducing at least one sign or symptom of a disease or disorder involving PIPKγ activity or cadherin localization in a patient (e.g., tumor or wound size).

Pharmaceutical compositions can be in the form of pharmaceutically acceptable salts and complexes and can be provided in a pharmaceutically acceptable carrier and at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically-acceptable carrier, composition or vehicle, such as a liquid or solid filler, diluent, excipient, or solvent encapsulating material, is involved in carrying or transporting the subject compound from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Pharmaceutical compositions appropriately formulated for parenteral (for example, by intravenous, intraperitoneal, subcutaneous or intramuscular injection), topical (including buccal and sublingual), oral, intranasal, intravaginal, or rectal administration can be prepared according to standard methods.

The selected dosage level will depend upon a variety of factors including the activity of the particular agent employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of an agent at levels lower than that required in order to achieve the desired therapeutic effect and gradually increase the dosage until the desired effect is achieved. Moreover, given the efficacy of an VEGFR1 siRNA developed by Sirna Therapeutics (San Francisco, Calif.) for the treatment of AMD, one of skill in the art can appreciate dosing of siRNAs useful for achieving the desired therapeutic result with no systemic or local adverse events.

To facilitate the identification of a patient having a cancer derived from epithelial cells (e.g., breast, colon, ovarian and prostate cancers) and the survival of such a patient, the present invention further relates to a method for diagnosing or prognosing a cancer via determining the amount and subcellular location of PIPKIγ. The diagnostic and prognostic method of the present invention involves the steps of obtaining a biological sample from a mammal; contacting the biological sample with an agent which specifically binds a PIPKIγ; determining the amount and subcellular location of the PIPKIγ; and comparing the amount and subcellular location of PIPKIγ in the biological sample to the amount and subcellular location of PIPKIγ in a reference sample. In the context of the instant invention, the term prognosis is intended to encompass predictions concerning patient survival. The diagnostic and prognostic method of the invention are intended to be used clinically in making decisions concerning treatment modalities, including therapeutic intervention, diagnostic criteria such as disease staging, and disease monitoring and surveillance for metastasis or recurrence of disease.

As used herein, a biological sample can be a tissue or biopsy sample isolated from a patient having or suspected of having a cancer. In contacting the biological sample with an agent which specifically binds PIPKIγ, the agent must be able to allow for the quantification and localization of PIPKIγ. Suitable agents include, antibodies, antisera or binding proteins which specifically interact with PIPKIγ and can be directly detected or labeled for detection. An antibody can be either a polyclonal or monoclonal antibody, e.g., as described by herein. Detection of an antibody can be realized by direct labeling of the antibody itself, with labels including a radioactive label such as ³H, ¹⁴C, ³⁵S, ¹²⁵I or ¹³¹I; a fluorescent label; a hapten label such as biotin; or an enzyme such as horse radish peroxidase or alkaline phosphatase. Alternatively, unlabeled primary antibody is used in conjunction with labeled secondary antibody such as antisera, polyclonal antisera or a monoclonal antibody specific for the primary antibody.

Detection, quantification, and localization of PIPKIγ is desirably carried out using any standard immunohistochemical staining and/or a binding assay well-known in the art or as disclosed herein. The amount of PIPKIγ present in the sample is subsequently compared to the amount of PIPKIγ present in a reference sample, e.g., a sample representing a known disease or health status, so that a diagnosis or prognosis can be made. It is contemplated that the comparison can be based on relative amounts or based on an index of amounts. The reference sample referred to is desirably from a subject not having a cancer and or from a subject having a particular stage of a cancer derived from epithelial cells. Alternatively, a reference sample can be from the patient, wherein one or more samples are taken over a period of time to establish progress or decline of the patient as to the disease.

A positive diagnosis of a cancer in a patient can be made, for example, when there is negative membrane staining for PIPKIγ in a biological sample from the patient, as compared to PIPKIγ staining of a reference sample isolated from a healthy individual. Likewise, an increased disease-specific survival prognosis can be made for a patient when, for example, high levels of membrane staining for PIPKIγ are found in a tumor-containing biological sample from the patient as compared to PIPKIγ staining in a reference sample isolated from an individual with a low chance of disease-specific survival.

The invention is described in greater detail by the following non-limiting examples.

### EXAMPLE 1

Constructs and Antibodies

C-terminus of E-cadherin was amplified by PCR and constructed into normal or modified (Ling, et al. (2002) supra) pET28 to generate His-tagged E-cadherin tail or HR-E-cadherin tail, which were then subcloned into pCMV-myc vector (Clontech, Palo Alto, Calif.). Wild-type E-cadherin, E-cadherinΔp120ctn, E-cadherinΔβctn, and E-cadherin/αctn are known in the art (Gottardi, et al. (2001) J. Cell Biol. 153:1049-1060). E-cadherin836 was amplified by PCR and E-cadherin Val832Met was generated using a QUIKCHANGE® II Site-Directed Mutagenesis Kit (STRATAGENE®, La Jolla, Calif.) according to manufacturer's instructions. Other mutants were generated using the following mutagenic primers and their complements: IγS645F, 5′-GGA GCT GGG TGT ACT TCC CGC TTC ACT ATA GC-3′ (SEQ ID NO:17); IγP646F, 5′-GGA GCT GGG TGT ACT CCT TCC TTC ACT ATA GCG CG-3′ (SEQ ID NO:18); IγL647V, 5′-GGG TGT ACT CCC CGG TTC ACT ATA GCG C-3′ (SEQ ID NO:19); and IγP646R, 5′-GCT GGG TGT ACT CCC GGC TTC ACT ATA GCG C-3′ (SEQ ID NO:20). The IγY644F mutant is known in the art (Ling, et al. (2003) supra). The full-length murine μ2-subunit yeast 2-hybrid clone was obtained from a murine brain cDNA library (Molecular Interaction Facility, University of Wisconsin, Madison, Wis.). A soluble truncation of this subunit was generated by digestion of the μ2-subunit open reading frame with an internal EcoRI site and an external 5′ XhoI site. The resulting fragment was cloned into pET28 and pET42 bacterial expression vectors (EMD Biosciences).

Monoclonal antibodies for E-cadherin (recognizing the C-terminus), N-cadherin, human VE-cadherin, p120catenin, β-catenin, γ-adaptin (C-8) and FITC-conjugated anti-E-cadherin were from TRANSDUCTION LABORATORIES™ (San Jose, Calif.). Polyclonal PIPKIγ anti-serum was generated according to established methods (Ling, et al. (2003) J. Cell Biol. 163:1339-1349). Regular mouse and rabbit IgG and secondary antibodies were from Jackson Immunoresearch Laboratories Inc. (West Grove, Pa.). Anti-HA (16B12) antibody was from COVANCE (Harrogate, UK). Anti-Myc, and FITC-conjugated anti-myc antibodies were from Upstate Biotechnology (Lake Placid, N.Y.). HRP-conjugated anti-GST antibodies were from Amersham Pharmacia Biotech (Piscataway, N.J.) and HRP-conjugated and monoclonal anti-T7 antibodies were obtained from NOVAGEN. Monoclonal anti-talin antibody (8d4) was purchased from Sigma (St. Louis, Mo.). Monoclonal anti-phosphotyrosine antibody (4G10) was purchased from Upstate. For Immunoblot analysis, monoclonal anti-E-cadherin antibody recognizing the ectodomain was obtained from ZYMED (South San Francisco, Calif.), whereas rat monoclonal anti-E-cadherin antibody for immunofluorescence was from Sigma (St. Louis, Mo.).

### EXAMPLE 2

Cell Culture and Transfection

HEK293, HeLa, and MDCK-TetOff cells (CLONTECH, Palo Alto, Calif.) and HEK293 cells were cultured in Dulbecco's modified eagle medium (Mediatech, Inc., Herndon, Va.) supplemented with 10% fetal bovine serum (INVITROGEN™, Carlsbad, Calif.). CHOIIIA cells, stably expressing hE/Fc were cultured in CHOIIIA medium (INVITROGEN™, Carlsbad, Calif.) supplemented with 10% dialyzed fetal bovine serum (INVITROGEN™, Carlsbad, Calif.) with non-essential amino acids. MDCK cells were transfected using FUGENE™ 6 (Roche Diagnostics, Indianapolis, Ind.) for 48 hours, then 100 μg/mL hygromycin B was added to the medium to select stable clones and 10 mg/mL doxycycline was utilized to suppress PIPKIγ expression. To induce expression, doxycycline was removed for 72 hours. HEK293 cells were transfected using the well-established calcium phosphate-DNA co-precipitation method for 48 hours. For siRNA knockout, MDCK cells in a 6-well plate were transfected twice for 48 hours intervals with 5 pmol/well siRNA using the calcium phosphate-DNA coprecipitation method. Forty-eight hours post the second transfection, cells were fixed and stained for immunofluorescence or immunoblotting.

### EXAMPLE 3

Immunoprecipitation, GST Pull-Down Assay and Immunoblotting

Immunoprecipitation was performed according to standard methods (Ling, et al. (2002) supra). Briefly, cells were harvested and lysed in lysis buffer (50 mM Tris-HCl, pH 7.5, 150 mM NaCl, 0.2% NP-40, 2 mM Na₃VO₄, 1 mM EDTA, 1 mM PMSF, and 10% glycerol). Cell lysates were incubated with 50 μL of 1:1 diluted Protein A-SEPHAROSE and 2-5 μg of the appropriate antibodies at 4° C. for 4 hours to overnight. The immunocomplexes were separated by 7.5% to 10% SDS-PAGE, and analyzed as indicated. GST-tagged PIPKIα, PIPKIγ635, PIPKIγ661, His-tagged E-cadherin tail or HR-E-cadherin tail were expressed and purified from E. coli, and GST pull-down assays were performed according to well-established methods (Ling, et al. (2003) supra). Briefly, recombinant T7-tagged PIPKIγ was incubated with GST-μ2 together with Glutathione SEPHAROSE 4 FAST FLOW (Amersham Biosciences) in 500 μl buffer B (PBS, 1% BSA, 0.4% TRITON X-100, and 2 mM DTT) for 4 hours or overnight at 4° C. The beads were washed with 1 ml buffer B four times, resolved by SDS-PAGE and analyzed via western blot. GST was used as a negative control for non-specific binding. All other GST pulldowns were performed with the proteins indicated in the same manner.

### EXAMPLE 4

Calcium Depletion, Surface Biotinylation and Trafficking of E-Cadherin

Cells were allowed to grow on coverslips for 72 hours to reach confluence and subsequently incubated with 2 mM EGTA for 20 minutes before being submitted to indirect immunofluorescence. Confluent MDCK cells grown in TRANSWELL® (COSTAR® Corporation, Cambridge, Mass.) were biotinylated by 1 mg/mL sulfo-NHS-SS-biotin (Pierce, Rockford, Ill.) and analyzed according to standard protocols (Le, et al. (1999) J. Cell Biol. 146:219-232). Internalization of E-cadherin was induced by 0.5 mM EGTA at 18° C. for indicated times. To measure the recycling of E-cadherin, MDCK cells were treated with 2 mM EGTA for 40 minutes at 37° C., chased in normal medium and surface biotinylation performed.

### EXAMPLE 5

Purification of hE/Fc and Adhesion Assay

hE/Fc was expressed and purified from the conditional culture medium of CHOIII cells stably expressing hE/Fc (Yap, et al. (1998) supra). Purified protein was kept in sterilized Tris-Ca buffer (50 mM Tris, 150 mM NaCl, 2 mM CaCl₂, pH 7.4). MDCK cells were treated with enzyme-free cell disassociation buffer (INVITROGEN™, Carlsbad, Calif.), washed twice with PBS, resuspended in serum-free medium and seeded on 10 μg/mL hE/Fc-coated coverslips for 1 hour, and then fixed for indirect immunofluorescence.

### EXAMPLE 6

Protein Expression and Purification in E. coli

Constructs in pET28 or pET42 expression vectors were transformed into BL21 (DE3) competent cells (NOVAGEN). Proteins were expressed and purified using His-BIND Resin following the manufacturer's instructions (NOVAGEN) or using Glutathione SEPHAROSE 4B FAST FLOW as per the manufacturer's instructions (Amersham Biosciences). Tyrosine phosphorylated recombinant PIPKIγ661 was generated via coexpression with Src and purified using established methods (Bairstow, et al. (2005) supra).

### EXAMPLE 7

PIP Kinase Activity Assay

Activity of purified recombinant PIPKIγ proteins was assayed against 25 μM PI4P micelles according to established methods (Ling, et al. (2002) supra).

### EXAMPLE 8

Transferrin Uptake Assays

Stable MDCK cells were grown in 10-cm dishes. Expression of exogenous PIPKIγ was induced for 72 hours by withdrawing doxycycline from the medium. For transferrin uptake assays, cells were incubated with serum-free medium for 2 hours. The serum-starved cells were then incubated with ALEXA FLUOR 647 transferrin (5 μg/ml, INVITROGEN) in binding medium at 37° C. for 20 minutes. After incubation, the cells were washed three times with PBS, three times with ice cold acid (0.2 M acetic acid and 0.5 M NaCl, pH 4.1), and three times with PBS again. Cells were finally collected by trypsinization and washed once with PBS. Half of the cells were used to determine PIPKIγ expression by flow cytometry. Briefly, cells were first incubated with primary anti-HA antibody and then with FITC-labeled secondary antibody. The fluorescence intensities of FITC staining were detected from approximately 10,000 cells and used to determine PIPKIγ expression. The other half of the cells was suspended in 0.5 ml PBS for determination of transferrin uptake using a FACSCALIBUR (BD Biosciences) flow cytometer. Fluorescence intensities of internalized ALEXA FLUOR 647 transferrin were quantified from approximately 10,000 cells.

### EXAMPLE 9

RNA interference

HeLa cells were maintained in DMEM containing 10% FBS. The cells were passed into 60-mm dishes one day prior to transfection. The cells were transfected with a PIPKIγ specific siRNA oligo using OLIGOFECTAMINE (INVITROGEN) transfection reagent. Duplexes of siRNA oligos (for both human and mouse: 5′-AAG UUC UAU GGG CUG UAC UGC-3′, SEQ ID NO:14; 5′-AAG GAC CUG GAC UUC AUG CAG-3′, SEQ ID NO:15; for canine: 5′-GAA GGC UCU UGU UCA CGA U-3′, SEQ ID NO:16) were obtained from Dharmacon (Lafayette, Colo.). Scrambled control siRNA (5′-AAG UAC CUG UAC UUC AUG CAG; SEQ ID NO:21) or PIPKIα specific siRNA (5′-AAG AAG UUG GAG CAC UCU UGG; SEQ ID NO:22) were used as controls. After 24 hours, the cells were transfected again in the same manner. The cells were then used for tranferrin uptake assays 72 hours post-transfection.

### EXAMPLE 10

Metabolic Labeling and Determination of Cellular PI4,5P₂ Levels

MDCK cells were metabolically labeled with 20 μCi/mL of H-myo-inositol (PERKINELMER) and the lipids were extracted and deacylated using established methods (Serunian, et al. (1991) supra). The deacylated glycerophosphoinositol phosphates were resuspended in water prior to analysis by HPLC. The deacylated lipids were separated using a ZORBAX SAX column and a gradient of 1.3 M ammonium phosphate (pH 3.85). The level of cellular PI4,5P₂ was determined with a PACKARD in-line liquid scintillation flow detector using deacylated ³H-PI4,5P₂ (PERKINELMER) as a standard.

### EXAMPLE 11

Immunofluorescence and Microscopy

Cells were washed with PBS at room temperature, fixed with 4% paraformaldehyde in PBS at room temperature for 15 minutes, and permeabilized with 0.2% TRITON X-100 in PBS at room temperature for 10 minutes. The cells were blocked with 3% BSA in PBS at room temperature for 1 hour, incubated with the primary antibody for 1 hour at 37° C. and washed with 0.1% TRITON X-100 in PBS. The cells were then incubated with fluorophore-labeled secondary antibody at room temperature for 45 minutes and washed with 0.1% TRITON X-100 in PBS. The coverslips were mounted to glass slides in VECTASHIELD (Vector Laboratories) mounting medium. Fluorescent images were captured using a NIKON ECLIPSE TE2000-U microscope with a COOLSNAP CCD camera (RS Photometrics) or using a 60× Plan oil immersion lens on a confocal laser-scanning microscope (model MR-1000; BIO-RAD Laboratories) mounted transversely to an inverted microscope (DIAPHOT 200, NIKON). Images were processed using PHOTOSHOP CS (Ling, et al. (2002) supra). To visualize the colocalization between AP2 and PIPKIγ661 constructs, MDCK cells in 6-well plates were transfected with 1 μg of each expression vector. After 16 hours of expression, the cells were incubated in serum-free DMEM for 2 hours and then treated with 50 μg/mL of transferrin (INVITROGEN) for 20 minutes at 37° C. The cells were subsequently fixed and stained as described herein.

### EXAMPLE 12

Immunohistochemistry and Tissue Array

Three hundred and sixty-nine sequential archival cases of invasive breast carcinoma that had undergone treatment at Vancouver General Hospital between 1974 and 1995 were identified for tissue microarray (TMA) construction. Median patient follow-up was 15 years. All patients had newly diagnosed invasive breast cancer and none presented with distant metastatic disease. TMA construction was carried out utilizing a tissue-arraying instrument (Beecher Instruments, Silver Springs, Md.) according to standard methods (Kononen, et al. (1998) Nat. Med. 4:844-847; Makretsov, et al. (2003) Hum. Pathol. 34:1001-1008). A monoclonal anti-E-cadherin antibody (clone C-36, TRANSDUCTION LABORATORIES) was used and PIPKIγ immunostaining was performed with polyclonal antibody recognizing all of isoforms.

All stained sections were scanned with a Baccus Laboratory Image Scanning System and a digital archive of all pathologic data was created for scoring. E-cadherin staining was scored as: negative (0), weak membranous (1), strong membranous (2), cytoplasmic (3), nuclear (4). PIPKIγ staining was scored as: negative (0), weak membranous (1), strong membranous (2), and cytoplasmic (3). All data were logged into a standardized score sheet matching each TMA section and then processed utilizing TMA-Deconvoluter 1.07 software. The data was then analyzed by the SPSS statistical software package (SPSS Version 11.0; SPSS, Chicago, Ill.). Correlation analysis was performed utilizing the 2-tailed Spearman non-parametric correlation test.

What is claimed is:

1. A method for identifying an agent that modulates the trafficking or binding activity of a PIPKIγ comprising contacting a PIPKIγ with a test agent in the presence of a cadherin or a μ-subunit and determining whether the agent modulates trafficking of the PIPKIγ or the cadherin, or binding of the PIPKIγ with the cadherin or the μ-subunit, as compared to a control, wherein a change in the trafficking or binding as compared to the control is indicative of the agent modulating the trafficking or binding activity of a PIPKIγ.

2. A method for diagnosing or prognosing a cancer comprising contacting a biological sample with an agent which specifically binds a PIPKIγ; determining the amount and subcellular location of the PIPKIγ; and comparing the amount and subcellular location of PIPKIγ in the biological sample to the amount and subcellular location of PIPKIγ in a reference sample, wherein the amount of membrane-localized PIPKIγ in the biological sample as compared to the reference sample is a diagnostic or prognostic indication of cancer.

3. A method for preventing or treating a disease or condition involving PIPKγ activity or cadherin localization comprising administering to a subject having or at risk of having a disease or condition involving PIPKγ activity or cadherin localization an effective amount of an agent which modulates the expression or trafficking or binding activity of a PIPKIγ thereby preventing or treating the disease or condition.
